(19) United States
(12) Patent Application Publication
(10) Pub. No.: US 2026/0258054 A1
(43) Pub. Date: Sep. 3, 2026

(54) SMALL MOLECULE FOR TREATMENT OF CANCER OF THE APPENDIX

(71) Applicants: The Regents of the University of California, Oakland, CA (US); Board of Regents, The University of Texas System, Austin, TX (US)

(72) Inventors: Dionicio Siegel, La Jolla, CA (US); Joshua Schweer, La Jolla, CA (US); Ruben Abagyan, La Jolla, CA (US); John Paul Shen, Austin, TX (US)

(73) Assignees: The Regents of the University of California, Oakland, CA (US); Board of Regents, The University of Texas System, Austin, TX (US)

(21) Appl. No.: 18/994,596

(22) PCT Filed: Jul. 17, 2023
(86) PCT No.: PCT/US2023/027939

§ 371 Date: Jan. 14, 2025

Related U.S. Application Data
(60) Provisional application No. 63/389,793, filed on Jul. 15, 2022.

(51) Int. Cl. C07D 519/00; A61K 31/522; A61K 31/675; A61P 35/00; C07D 473/18; C07F 9/6561

(52) U.S. Cl. CPC C07D 519/00; A61K 31/522; A61K 31/675; A61P 35/00; C07D 473/18; C07F 9/65616

(57) ABSTRACT

This disclosure provides compounds, compositions and methods for one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell in a subject in need thereof by administering to the subject an effective amount of a compound or composition of this disclosure. Also provided are compounds, compositions and methods for treating cancer in a patient in need thereof, by administering to the subject an effective amount of a compound or composition as disclosed herein.

### CROSS-REFERENCE TO RELATED APPLICATION

This application claims the benefit under 35 U.S.C. § 119 (e) of U.S. Provisional Application Ser. No. 63/389,793, filed Jul. 15, 2022, which is incorporated by reference in its entirety.

### BACKGROUND

New treatments are needed for appendiceal cancer, currently an orphan disease. Appendiceal tumors such as appendiceal adenocarcinoma (AA) encompass a rare and diverse group of neoplasms; AA is the most common histologic subtype. Epidemiologic studies based on Surveillance, Epidemiology, and End Results (SEER) data have shown a steady increase in incidence from approximately 0.2 cases per 100,000 in the 1970s, to current estimates of just over 1 per 100,000. In comparison, this is 40-fold less common than colon cancer, which in the US has an incidence of approximately 40 per 100,000. Cases of early-onset AA, defined as diagnosis before age 50, have increased by 24% between 2011 to 2016, and in 2016 represented 40% of all appendiceal cancer. In contrast, the increase in early-onset colorectal cancer (CRC) was only 2.2% for that same time period. Historically, appendiceal tumors have been grouped together with CRCs, and as of 2021 the National Comprehensive Cancer Network (NCCN) guidelines still suggested that appendiceal tumors be treated with chemotherapy similarly to colon tumors. The rarity of AA has made it difficult to conduct clinical trials, and in the absence of trial data, the NCCN guidelines assume biological similarity due to anatomic vicinity, common embryological origin, and common expression of the transcription factor CDX2. However, there is a growing consensus that AA is a clinically and molecularly distinct entity from CRC, and that AA specific therapies (none exist currently) need to be developed. This disclosure satisfies this need and provides related advantages as well.

### SUMMARY OF THE DISCLOSURE

The Ga protein GNAS, which encodes for the heterotrimeric G protein Gαs, is the second most frequently mutated gene in mucinous appendiceal adenocarcinoma (AA) (~50% of tumors) and Pseudomyxoma Peritonei (PMP, ~75% of tumors) and third most common in non-mucinous AA (~25% of tumors), making it a promising drug target in this orphan disease. Although classically druggable, no commercially available inhibitors of Gαs currently exist. Applicant provides an innovative approach to develop and characterize chemical inhibitors of Gαs. Given prior in vitro and in vivo data demonstrating that GNAS knockout is lethal to GNAS^{R201} tumors, chemical inhibition of Gαs will be an effective therapeutic strategy for GNAS^{R201} mutant tumors.

The GNAS^{R201} gain-of-function mutation is the single most frequent cancer-causing mutation across all heterotrimeric G proteins, driving oncogenesis in various low-grade/benign gastrointestinal and pancreatic tumors. In one aspect of this disclosure, Applicant investigated the role of GNAS and its product Gαs in tumor progression using peritoneal models of colorectal cancer (CRC). GNAS was knocked out in multiple CRC cell lines harboring GNAS^{R201C/H} mutations (KM12, SNU175, SKCO1), leading to decreased cell-growth in 2D and 3D organoid models. Nude mice were peritoneally injected with GNAS-knockout KM12 cells, leading to a decrease in tumor growth and drastically improved survival at 7 weeks. Supporting these findings, GNAS overexpression in LS174T cells led to increased cell-growth in 2D and 3D organoid models, and increased tumor growth in PDX mouse models. GNAS knockout decreased levels of cyclic AMP in KM12 cells, and molecular profiling identified phosphorylation of β-catenin and activation of its targets as critical downstream effects of mutant GNAS signaling. Supporting these findings, chemical inhibition of both PKA and β-catenin reduced growth of GNAS mutant organoids. Applicant's findings demonstrate oncogene addiction to GNAS in peritoneal models of GNAS^{R201C/H} tumors, which signal through the cAMP/PKA and Wnt/β-catenin pathways. Thus, GNAS and its downstream mediators are therapeutic targets for GNAS mutant tumors.

Thus, in one aspect, this disclosure provides compounds and compositions that are useful therapeutically and in investigational studies. In one aspect, provided are compounds of Formula (I)

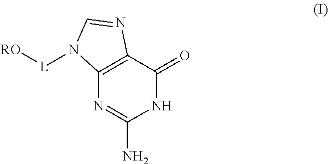

or a pharmaceutically acceptable salt thereof;
wherein:
R is H,

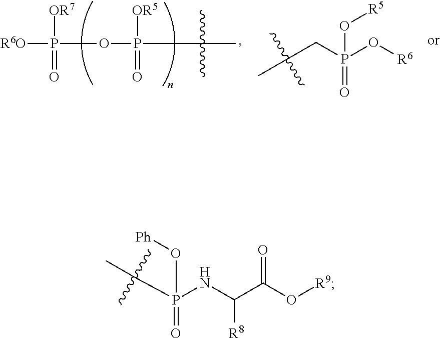

L is

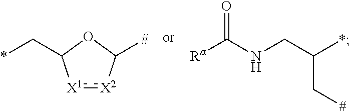

* denotes the linkage point with the —OR group and #denotes the linkage point with the heterocycle;
X¹ is CR¹ or CR¹R²;
X² is CR³ or CR³R⁴;
- - - is absent or a single bond, provided that when - - - is a single bond, X¹ is CR¹ and X² is CR³;
R¹, R², R³, and R⁴, when present, are each independently H, OH, SH, CN, C₁-C₆ alkyl, C₁-C₆ alkoxy, a —O-sulfolnyl group, or halide; or any two of R¹, R², R³, and R⁴ are taken together with the carbon atoms to which they are bound to form a ring having 3-6 ring atoms comprising a moiety selected from the group consisting of O, S, SO, SO₂, and O(SO₂)O, or if on the same carbon atom, to form an oxo (═O) group or an optionally substituted alkenyl group;
R^{a} is optionally substituted alkenyl or haloalkyl;
R⁵, R⁶, and R⁷ are each independently absent, H, or —(CH₂)—O—(CO)—(C₁-C₆ alkyl);
R⁸ and R⁹ are each independently H or C₁-C₆ alkyl; and
n is 0, 1 or 2.

In a further aspect of the above compound provided are compounds having a structure of Formula (II)

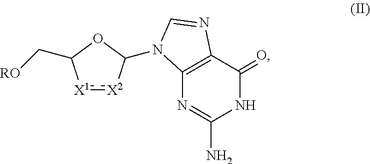

or a pharmaceutically acceptable salt thereof.

In another aspect of the above compound provided are compounds having a structure of Formula (IIa)

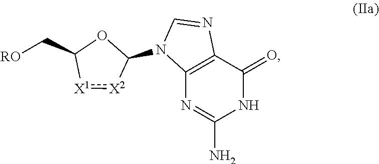

or a pharmaceutically acceptable salt thereof.

In another aspect of the above compound provided are compounds having a structure of Formula (III)

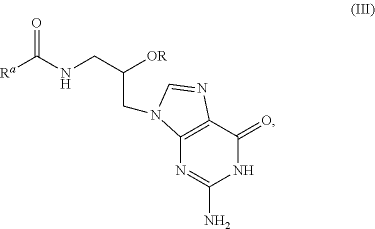

or a pharmaceutically acceptable salt thereof.

In another aspect of the above compound provided are compounds having a structure of Formula (IIIa) or (IIIb)

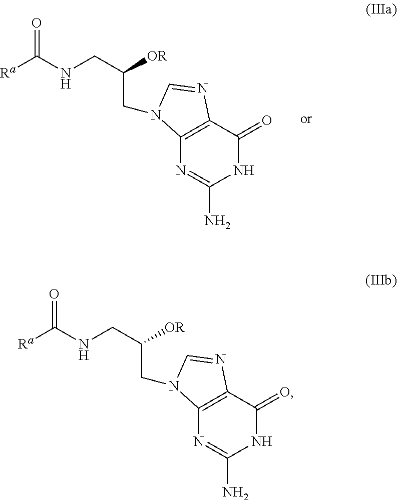

or a pharmaceutically acceptable salt thereof.

The compounds and compositions as disclosed herein are useful in methods for one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell, the method comprising contacting the cell with a compound or composition as disclosed herein. In one aspect the cell is a mammalian cancer cell. The contacting can be in vitro or in vivo.

Further provided are methods of one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell in a subject in need thereof, the method comprising, or consisting essentially of, or yet further consisting of administering to the subject an effective amount of a compound or composition as disclosed herein.

Also provided is a method of treating cancer in a patient in need thereof, the method comprising, or consisting essentially of, or yet further consisting of administering to the subject an effective amount of a compound or composition as disclosed herein. The cancer can be a primary cancer (localized) or metastatic, and thus selected from Stage I, Stage II, Stage III, or Stage IV. The therapy can be administered subsequent to resection of the cancer in the subject prior to or subsequent to administration.

Further aspects of the compounds, compositions and methods are disclosed herein.

### BRIEF DESCRIPTION OF THE DRAWINGS

FIGS. 1A-1D: Incidence of GNAS mutations in pan-cancer. (FIG. 1A), Frequency of GNAS mutations across various pan-cancers obtained from the cBioportal database and (FIG. 1B), estimated number of GNAS mutant cases in 2021 using USA cancer statistics. (FIG. 1C), Mutation map of GNAS protein. Lollipops along the domain structure of the protein represent the total frequencies of all mutated alleles of GNAS in a population. Diagram circles represent different GNAS variants in 1050 pan-cancer samples (*=Truncating mutations (Nonsense, Frameshift insertion/deletion), #=Other types of mutations, others=Missense mutations). The x-axis and γ-axis represent the amino acid number and the frequency of the mutation, respectively. Mutation maps were generated using the web-based tool MutationMapper for plotting lollipop plot available in cBioPortal. (FIG. 1D), Co-mutation network of all GNAS^{R201} variants with other mutant genes in colorectal cancer samples from the institutional molecular database of The University of Texas MD Ander Cancer Center. Edge weights in the network were scaled with respect to the magnitude of odds ratio of the Chi-square test performed between the pair of mutated genes.

FIGS. 2A-2E: Evaluation of GNAS knockout and overexpressing CRC cell lines. (FIG. 2A), GNAS was knocked out from KM12, SNU175, and SKCO1 cell lines using a CRISPR-mediated approach, and its effect on clonogenic capacity was assessed by evaluating growth in standard culture conditions for 12-15 days. Colonies were fixed in 1% paraformaldehyde and stained with 1% crystal violet prior to counting (mean±SD, n=5, 4, 4 respectively). (FIG. 2B), GNAS was overexpressed in the LS174T cell line using a doxycycline inducible promoter, and the resulting effect on clonogenic capacity was assessed as previously described (mean±SD, n=3 in each group) (Franken N A, Rodermond H M, Stap J, Haveman J, van Bree C. Clonogenic assay of cells in vitro. Nat Protoc. 2006; 1(5):2315-9). (FIG. 2C), Growth of GNAS^{KO} SNU175 cells (number of colonies, total colony area) evaluated with a 3D organoid assay. (FIG. 2D), A single clone (GNAS^{352delG}) was selected from a pool of GNAS^{KO} cells, and growth (number of colonies, total area) was evaluated with a 3D organoid assay. (FIG. 2E), Growth of GNAS^{R201} overexpressing LS174T cell lines evaluated with a 3D organoid assay (mean±SD, n=3 in each group).

FIGS. 3A-3F: GNAS promotes tumor formation from KM12 and LS174T cells in the peritoneum of NSG mice. (FIG. 3A), NSG mice were intraperitoneally injected with 10⁶ parental (GNAS^{R201H}) or GNAS-knockout (GNAS^{KO-pool}) KM12 cells. At 1 to 6 weeks post-injection, D-luciferin (150 mg/kg) was injected via IP and the bioluminescence signal was obtained using an in vivo imaging system (IVIS) with small binning and 1 sec exposures at 10 min after administration of luciferin. Three representative merged images are shown. (FIG. 3B), Data expressed as mean±SEM, n=6 in each group. (FIG. 3C), Survival curve of NSG mice injected with KM12 cells (GNAS^{R201H} or GNAS^{KO-pool}). (FIG. 3D), Tissue sections of mouse tumors were stained with H&E. 1× bar, 3 mm (left) and 4× bar, 600 μm (right). (FIG. 3E), NSG mice were intraperitoneally injected with 2.5×10⁴ parental, GNAS^{WT}, GNAS^{R201C}, and GNAS^{R201H} overexpressing LS174T cells. Bioluminescent signal was obtained 1 to 6 weeks post-injection as described in A, and four representative images are shown. (FIG. 3F), Data are expressed as mean±SEM, n=3 in each group. *P<0.05, **P<0.01, ***P<0.001.

FIGS. 4A-4C: GNAS signals through the CAMP-PKA axis. (FIG. 4A), KM12 GNAS^{R201H} and GNAS^{352delG} cells were treated with forskolin (0-250 μM) for 15 min. Following the treatment, cells were lysed, and cAMP levels were measured as described in the methods. (FIG. 4B), LS174T GNAS^{R201H} cells were treated with and without doxycycline, and cAMP levels measured after induction with forskolin (0-2.5 μM) for 15 min. (FIG. 4C), KM12 GNAS^{R201H} and GNAS^{352delG} organoids were grown in 3D-matrigel domes and treated with the PKA inhibitor H-89 (60 μM) for 10 days.

FIGS. 5A-5F: β-catenin is a downstream mediator of mutant GNAS signaling. (FIG. 5A), Top 10 hallmark genesets from the KM12 GNAS^{KO-pool} and LS174T GNAS overexpression datasets were selected and overlapping genesets were plotted. (FIG. 5B), Ratio of pT41/S45 phosphoβ-catenin/β-catenin based on the RPPA analysis results. (FIG. 5C), Immunohistochemistry (IHC) staining of β-catenin in LS174T dox (−) and dox (+) tissues from intraperitoneal mouse tumors. (FIG. 5D), Quantification of IHC tumors showing % of cells staining positive of β-catenin. (FIG. 5E), Quantification of the cytoplasmic and nuclear distribution of β-catenin based on IHC staining. (FIG. 5F), Effect of LF3 (β-catenin inhibitor) on the growth of KM12 (GNAS^{R201H} and GNAS^{352delG}) organoids. DMSO (0.2%) was used as control treatment and organoids were cultured for 10 days.

FIGS. 6A-6G: Validation of GNAS knockout and overexpressing CRC cell lines. (FIG. 6A), Western blot showing GNAS protein knockdown and (FIG. 6B), RNA-sequencing showing GNAS mRNA decrease in KM12, SNU175, and SKCO1 cell lines. (FIG. 6C), Zygosity of GNAS^{R201} mutation from n=30 cBioPortal tumors determined by calculating the relative variant allele frequency (FIG. 6D), Western blot showing GNAS protein induction and (FIG. 6E), RNA-sequencing analysis showing increased GNAS mRNA expression in doxy inducible constructs. (FIG. 6F), Validation of GNAS knockout in the GNAS^{352delG} single clone by Sanger-sequencing and (FIG. 6G), western blot showing stronger effect GNAS^{352delG} knockout in comparison to GNAS^{KO-pool}.

FIGS. 7A-7D: GNAS primarily encodes for the Gαs short isoform. (FIG. 7A), Expression of all GNAS encoded transcripts (transcripts per million) identified by RNA-sequencing in all GNAS knockout cell-line constructs. (FIG. 7B), Expression of top 2 meaningfully expressed GNAS transcripts across cell-line constructs (FIG. 7C), Mapping of identified transcripts to UniProtKB IDs. (FIG. 7D), Expression of various GNAS encoded transcripts in a TCGA cohort of 460 colon adenocarcinoma tumors.

FIGS. 8A-8C: GNAS^{352delG} single clone knockout reduces tumor growth in mice. (FIG. 8A), NSG mice were intraperitoneally injected with 10⁵ parental GNAS^{R201H} or single-clone GNAS^{352delG} cells. Three representative merged images are shown (top). Data are expressed as mean±SEM, n=5 in each group (bottom). (FIG. 8B), Tumor nuclei were stained with Ki-67 and (FIG. 8C), quantified by measuring percentage of positive stained nuclei.

FIGS. 9A-9B: Wnt stimulation rescued growth of GNAS knockout organoids. (FIG. 9A), 3D growth of KM12 GNAS knockout organoids after supplementation with Wnt3a or R-spondin conditioned medium. (FIG. 9B), Quantification of growth by measuring total colony area (%).

FIG. 10: Mutation frequencies for most commonly mutated driver genes, separated by histologic subtypes (PMP=pseudomyxoma peritonei, Mad=mucinous adenocarcinoma, Ad=adenocarcinoma, SRCC=signet-ring cell carcinoma.

FIG. 11: GNAS mutation in AA, the concentration in the R201 codon is a classic pattern for an oncogenic, gain-of-function mutation. Left, proportions of specific GNAS mutations in AA.

FIG. 12: GNAS KO decreases colony formation, t-test p<0.0001 for each case. Western blot confirms loss of protein in GNAS KO (Right band) relative to wildtype.

FIGS. 13A-13C: In vivo tumor formation of GNAS isogenic cell lines. (FIG. 13A), Live imaging of mice injected into peritoneum with either GNAS isogenic KM12 (FIG. 13B), or LS174T cells. (FIG. 13C), Histology of KM12 GNAS isogenic tumors, note less mucin and fibrovascular stroma in GNAS KO.

FIGS. 14A-14D: GNAS activates Wnt pathway. (FIG. 14A), Images of in vitro organoid formation of KM12 GNAS isogenic cell lines, note Wnt agonists Wnt3A or R-spondin rescue GNAS knockout. (FIG. 14B), Quantitation of organoid assay. (FIG. 14C), Immunohistochemistry of isogenic Ls174T PDX tumor stained with anti-β-catenin, note increased staining when GNAS^{R201H} is expressed by adding doxycycline. (FIG. 14D), Ratio of phospho β-catenin to unphosphorylated β-catenin as measured by Reverse Phase Protein Array (RPPA) in KM12 cell lines (black) and PDX tumors (grey).

FIG. 15: Pre-clinical models of AA. Left: UMAP clustering of cell lines and tumors using gene expression data. Each shade denotes a tumor type (some labels removed for clarity). Note AA tumors cluster separately CRC and that AA PDX cluster with AA tumors. Middle: GSEA comparing Ls173T GNAS^{R201} grown in 2D culture vs. orthotopic PDX. Right: Histology of AA PDX showing a mucinous adenocarcinoma (AAPDX-01) and a colonic type (non-mucinous) adenocarcinoma.

FIG. 16: Structure of GDP bound to GNAS showing the proximity of C201. Structures of GDP and the new inhibitor GDP-epoxide. Proximity induced covalent reaction of cysteine 201 with GDP-epoxide.

FIG. 17: cAMP assay in KM12 cells, LS174T.

FIG. 18: Patient-derived micro-oganospheres (PDMOs). Cells from a CRC metastatic liver biopsy encapsulated in the droplet organosphere (350 uM diameter) rapidly form organoid structures within days, ready for drug testing.

FIG. 19: Schematic of drug testing in orthotopic appendiceal cancer PDX. Tumors are implanted into mice and imaged after four weeks (time zero) to establish pre-treatment baseline. Serial changes in tumor volume are measure by MRI, similar to a human clinical.

FIG. 20: JVS-324 inhibits cAMP production in GNAS mutant CRC cell line.

FIG. 21: JVS-324 inhibits cAMP production in CRC cell line expressing GNAS^{R201C} mutation.

FIG. 22: Effects of JVS-324 on viability in Ls174T cells and isogenic pair expressing GNAS^{R201C}.

FIG. 23: Effects of JVS-324 on SNU-175 cell line, which contains GNAS^{R201C} mutation.

FIG. 24: GE3 inhibits cAMP production in SNU-175 (GNAS^{R201C} CRC cell line).

FIG. 25: GE3 inhibits cAMP production in SNU-175 (GNAS^{R201C} CRC cell line).

FIG. 26: GE3 decreased viability of SNU-175 (GNAS^{R201C} CRC cell line).

### DETAILED DESCRIPTION

### Definitions

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. In one aspect, the term about intends variation of (+) or (−) of 0.5, or about 1.0, or about 1.5, or about 2.0, or about 2.5, or about 3.0, or about 3.5, or about 4.0, or about 4.5 or about 5.0 from the numerical designation, for example temperature. It also is to be understood, although not always explicitly stated, that the reagents described herein are merely exemplary and that equivalents of such are known in the art.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The phrase “and/or” as used in the present disclosure and claims will be understood to mean any one of the recited members individually or a combination of any two or more thereof—for example, “A, B, and/or C” would mean “A, B, C, A and B, A and C, B and C, or the combination of A, B, and C.”

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

The GNAS gene is located on the long arm of chromosome 20 in humans and gives rise to multiple gene products, including transcripts that encode the alpha-subunit of the stimulatory guanine nucleotide-binding protein (Gsα), extra-large Gsα (XLαs), and neuroendocrine secretory protein 55 (NESP55). Gsα is encoded by exons 1-13, while NESP55, XLαs, and A/B individually contain their own unique first exons that splice onto exon 2-13 of GNAS. The mature GNAS-AS1 transcript is spliced but is subject to alternative splicing.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer such as breast cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

As used herein, a biological sample, or a sample, is obtained from a subject. Exemplary samples include, but are not limited to, cell sample, tissue sample, tumor biopsy, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, an active ingredient (a.i.) or agent can be an anticancer agent. An anticancer agent refers to any drug or compound used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives thereof.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical c “Cryoprotectants” are known in the art and include without limitation, e.g., sucrose, trehalose, and glycerol. A cryoprotectant exhibiting low toxicity in biological systems is generally used.

“Composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include intraperitoneal administration, oral administration, nasal administration, injection, and topical application.

An agent of the present disclosure can be administered for therapy by any suitable route of administration. It will also be appreciated that the optimal route will vary with the condition and age of the recipient, and the disease being treated.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cₘ-Cₙ, such as C₁-C₁₂, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)—CH₂, among others. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C═CH, —C═CCH₃, —CH₂C═CCH₃, —C═CCH₂CH(CH₂CH₃)₂, among others. Alkynyl groups may be substituted or unsubstituted. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be mono-substituted or substituted more than once. For example, monosubstituted aryl groups include, but are not limited to, 2-, 3-, 4-, 5, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Aralkyl groups may be substituted or unsubstituted. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Heterocyclyl groups may be substituted or unsubstituted. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Heteroaryl groups may be substituted or unsubstituted. Thus, the phrase “heteroaryl groups” includes fused ring compounds as well as includes heteroaryl groups that have other groups bonded to one of the ring members, such as alkyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene. Such groups may further be substituted or unsubstituted.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

The terms “alkanoyl” and “alkanoyloxy” as used herein can refer, respectively, to —C(O)-alkyl and —O—C(O)-alkyl groups, where in some embodiments the alkanoyl or alkanoyloxy groups each contain 2-5 carbon atoms. Similarly, the terms “aryloyl” and “aryloyloxy” respectively refer to —C(O)-aryl and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylic acid” as used herein refers to a compound with a —C(O)OH group. The term “carboxylate” as used herein refers to a —C(O)O⁻ group. A “protected carboxylate” refers to a —C(O)O-G where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “ester” as used herein refers to —COOR⁷⁰ groups. R⁷⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “thiol” refers to —SH groups, while sulfides include —SR⁸⁰ groups, sulfoxides include —S(O)R⁸¹ groups, sulfones include —SO₂R⁸² groups, and sulfonyls include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “halogen”, “halo” or “halide” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—.

The term “nitro” as used herein refers to an —NO₂ group.

The term “epoxide” refers to a three-membered ring structure in which one of the vertices is an oxygen and the other two are carbons. For example, an epoxide moiety may be

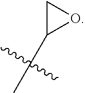 [SMILES: *C1CO1]

The term “sulfonyl” refers to a —S(═O)₂R′, or —S(═O)₂-group, where R′ is selected from hydrogen, alkyl, alkenyl, alkynyl, heteroalkyl (bonded through a chain carbon), amino, cycloalkyl, aryl, arylalkyl, heteroaryl (bonded through a ring carbon), heteroarylalkyl, heterocycloalkyl (bonded through a ring carbon), unless stated otherwise in the specification, each of which moiety can itself be optionally substituted as described herein. For example, in one embodiment, the sulfonyl group is —S(═O)₂R′, wherein R′ is alkyl substituted with a carbonyl group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

The following terms are used throughout as defined below. As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C14, P32 and S35 are thus within the scope of the present technology. Procedures for inserting such labels into the compounds of the present technology will be readily apparent to those skilled in the art based on the disclosure herein.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

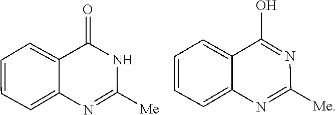

As another example, guanidines may exhibit the following isomeric forms in protic organic solution, also referred to as tautomers of each other:

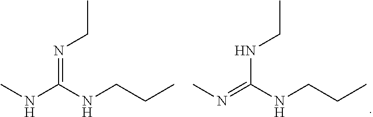

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

### Modes For Carrying Out The Disclosure

GNAS encodes for the G protein subunit Gαs—these cytosolic, membrane-bound, proteins regulate various intracellular signaling pathways in response to the activation of G protein-coupled receptors (GPCRs). Multiple studies have shown that GPCR-associated signaling is responsible for aberrant cell growth and proliferation, leading to cancer invasion, metastasis, and maintenance of the tumor microenvironment. When activated by a ligand, GPCRs undergo a conformational change that activates the heterotrimeric G protein; GTP is bound to the Ga subunit (Gas, encoded by GNAS) and the Ga subunit dissociates from the Gβγ subunit. R201 mutations in GNAS result in higher Gαs protein activity, leading to the activation of adenylate cyclase, increased cyclic-AMP, and the activation of various downstream oncogenic signaling pathways. Traditionally it was thought that the GNAS^{R201} mutation prevented hydrolysis of GTP to GDP, allowing for signaling in the absence of the GPCR ligand. However, it has been recently shown that GNAS^{R201C}, in addition to having reduced GTP hydrolysis, can also activate adenylate cyclase when it is in the GDP-bound state.

While GNAS signaling helps maintain cell differentiation in many tissue contexts, such gain-of-function mutations in GNAS have been shown to drive oncogenesis in various GI cancers. For instance, genetic studies have confirmed that mutations in the R²⁰¹ codon of GNAS drive oncogenesis in appendiceal, colon, and gastric adenocarcinoma, as well as Intraductal Papillary Mucinous Neoplasms (IPMN) of the pancreas and Small Cell Lung Cancer (SCLC). Moreover, GNAS is the second most frequently mutated gene in mucinous AA (~50% of tumors) and Pseudomyxoma Peritonei (PMP, ~75% of tumors) and third most common in non-mucinous AA (~25% of tumors), making it a promising drug target for this orphan disease.

Canonically, Gαs activation stimulates adenylyl cyclase, which leads to the subsequent accumulation of the secondary messenger cAMP. Protein Kinase A (PKA) and ePAC1/2 guanine-nucleotide exchange factors are the two primary targets of cAMP, and each has varying downstream functions. Wnt signaling, a key regulator of stemness, development, and carcinogenesis, is also hypothesized to be a downstream effector of constitutive Gαs signaling. However, the signaling mechanism downstream of Gαs is largely unclear, and like other GPCR and G proteins in likely to be tissue specific. Applicant evaluated the GNAS dependence of peritoneal tumors bearing activating GNAS^{R201} mutations and identify key signaling pathways downstream of Gαs thereby providing the disclosed composition and method for use in treating these tumors and cancers.

### Compounds and Compositions

This disclosure provides compounds and compositions that are useful therapeutically and in investigational studies. In one aspect, provided are compounds of Formula (I)

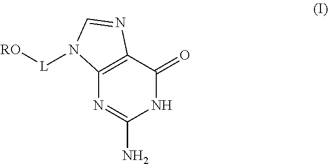

or a pharmaceutically acceptable salt thereof;
wherein:
R is H

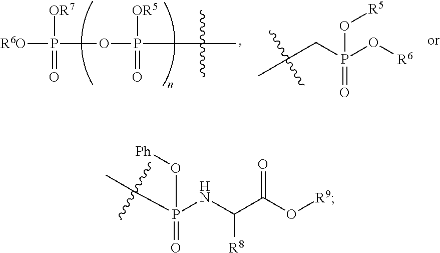

L is

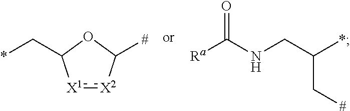

* denotes the linkage point with the —OR group and #denotes the linkage point with the heterocycle;
X¹ is CR¹ or CR¹R²;
X² is CR³ or CR³R⁴;
- - is absent or a single bond, provided that when - - - is a single bond, X¹ is CR¹ and X² is CR³;
R¹, R², R³, and R⁴, when present, are each independently H, OH, SH, CN, C₁-C₆ alkyl, C₁-C₆ alkoxy, a —O-sulfolnyl group, or halide; or any two of R¹, R², R³, and R⁴ are taken together with the carbon atoms to which they are bound to form a ring having 3-6 ring atoms comprising a moiety selected from the group consisting of O, S, SO, SO₂, and O(SO₂)O, or if on the same carbon atom, to form an oxo (═O) group or an optionally substituted alkenyl group;
R^{a} is optionally substituted alkenyl or haloalkyl;
R⁵, R⁶, and R⁷ are each independently absent, H, or —(CH₂)—O—(CO)—(C₁-C₆ alkyl);
R⁸ and R⁹ are each independently H or C₁-C₆ alkyl; and
n is 0, 1 or 2.

In some embodiments, the compound described herein is a compound having a structure of Formula (II)

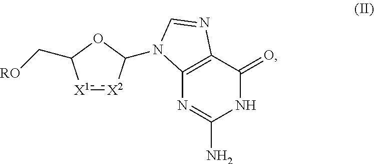

or a pharmaceutically acceptable salt thereof.

In some embodiments, the compound described herein is a compound having a structure of Formula (IIa)

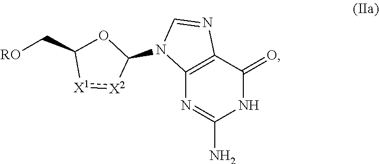

or a pharmaceutically acceptable salt thereof.

In some embodiments, the compound described herein is a compound having a structure of Formula (III)

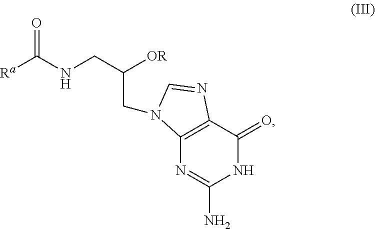

or a pharmaceutically acceptable salt thereof.

In some embodiments, some embodiments, the compound described herein is a compound having a structure of Formula (IIIa) or (IIIb)

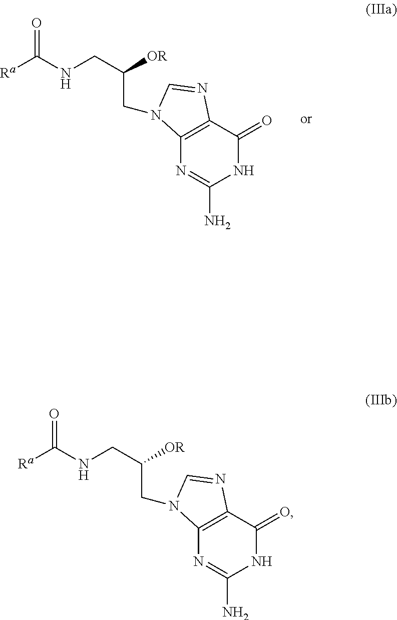

or a pharmaceutically acceptable salt thereof.

In any embodiment herein, it may be that - - - is absent.

In any embodiment herein, it may be that - - - is a single bond, and, X¹ is CR¹ and X² is CR³. In some embodiments, at least one of R¹ and R³ is not H.

In any embodiment herein, it may be that, R¹, R², R³, and R⁴, when present, are each independently H, OH, SH, a —O-sulfolnyl group, halide, or CN.

In any embodiment herein, it may be that the —O-sulfolnyl group is a mesylate group (i.e., —OSO₂Me).

In any embodiment herein, it may be that, R¹ and R³ are taken together with the carbon atoms to which they are bound to form a 5-membered ring comprising a O(SO₂)O moiety.

In any embodiment herein, it may be that R¹ and R³ are taken together with the carbon atoms to which they are bound to form an epoxide group.

In any embodiment herein, it may be that R¹ and R², or R³ and R⁴ are taken together with the carbon atom to which they are bound to form an epoxide group.

In any embodiment herein, it may be that R¹ and R², or R³ and R⁴ are taken together with the carbon atom to which they are bound to form an oxo (═O) group or an optionally substituted alkenyl group (e.g., an optionally substituted ═CH₂ group).

In any embodiment herein, it may be that the alkenyl group is a ═CH₂ group.

In any embodiment herein, it may be that R^{a} is an alkenyl optionally substituted with an amino group, or a haloalkyl. In any embodiment herein, it may be that R^{a} is

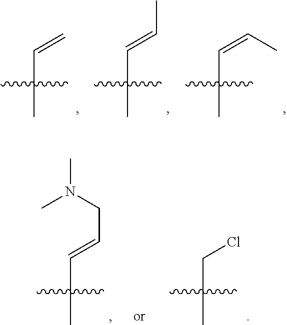

In any embodiment herein, it may be that n is 0 or 1. In embodiments, n is 0. In embodiments, n is 1.

In any embodiment herein, it may be that R¹, R², R³, and R⁴, when present, then at least one of R¹, R², R³, and R⁴ is not H.

In any embodiment herein, it may be that R¹, R², R³, and R⁴, when present, then at least one of R¹ and R² is not H, and, at least one of R³ and R⁴ is not H.

In any embodiment herein, it may be that R is H. In any embodiment herein, it may be that R is

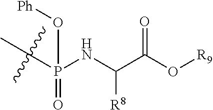

wherein R⁸ and R⁹ are each independently a C₁-C₆ alkyl

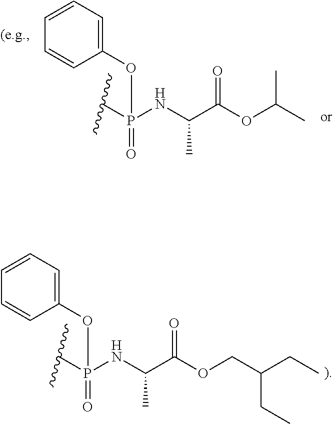 [SMILES: *P(=O)(N[C@@H](C)C(=O)OC(C)C)Oc1ccccc1 ; *P(=O)(N[C@@H](C)C(=O)OCC(CC)CC)Oc1ccccc1]

In any embodiment herein, it may be that R is H, PO₃²⁻, PO₃PO₃³⁻,

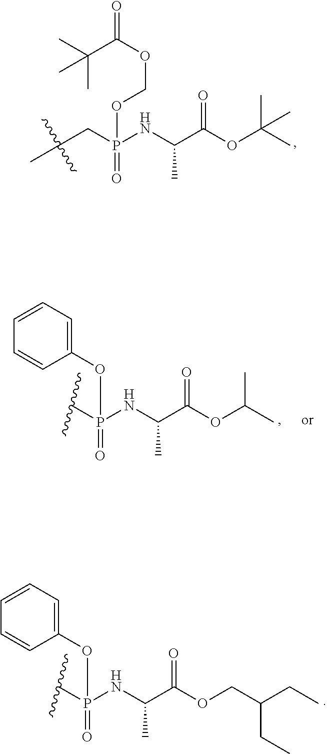 [SMILES: *CP(=O)(N[C@@H](C)C(=O)OC(C)(C)C)OCOC(=O)C(C)(C)C ; *P(=O)(N[C@@H](C)C(=O)OC(C)C)Oc1ccccc1 ; *P(=O)(N[C@@H](C)C(=O)OCC(CC)CC)Oc1ccccc1]

In any embodiment herein, it may be that the compound described herein is a compound having the following structures:

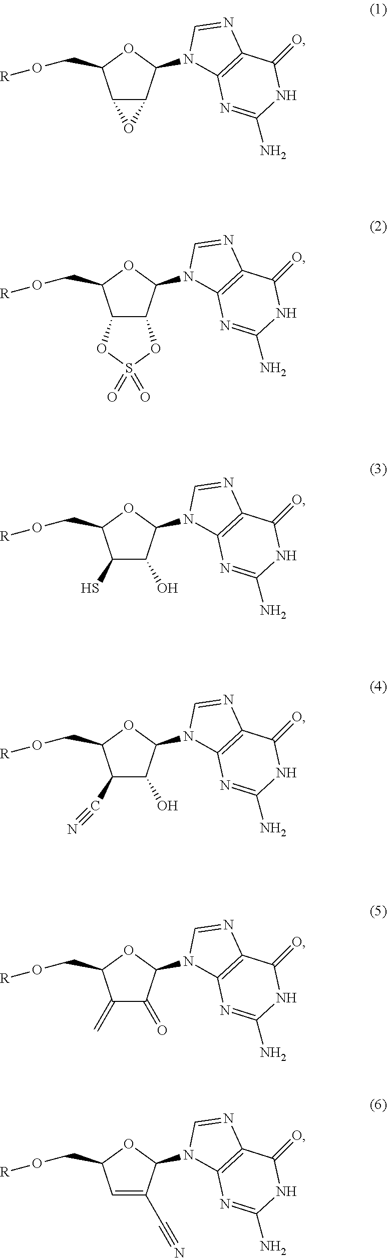

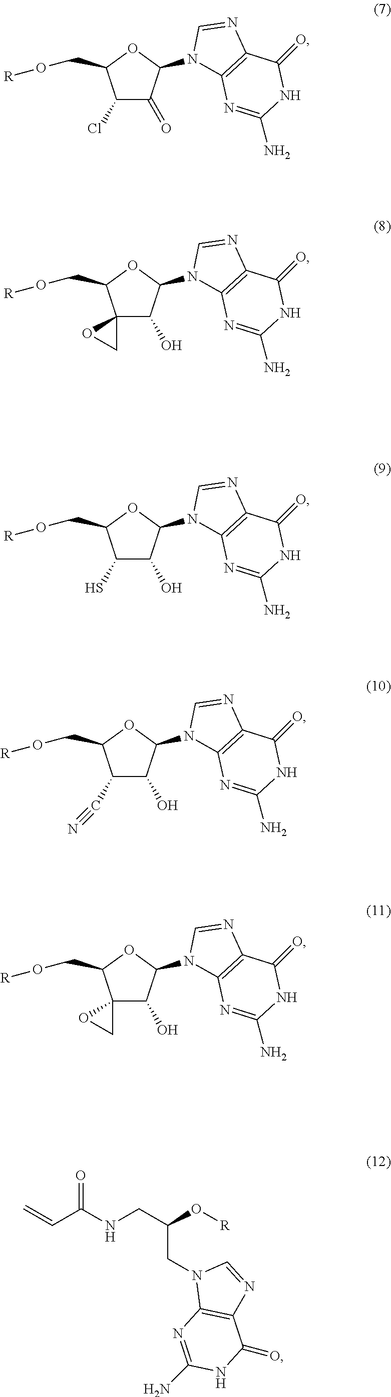

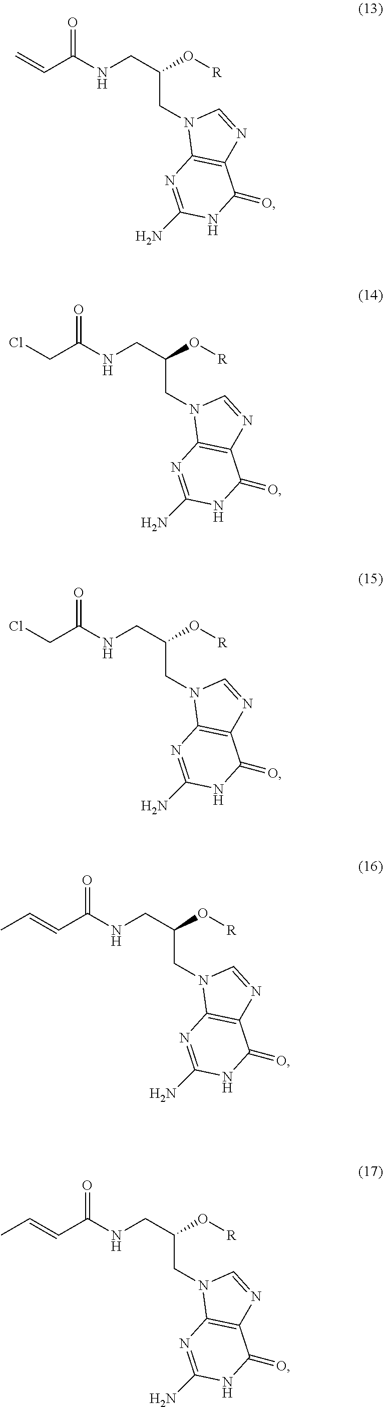

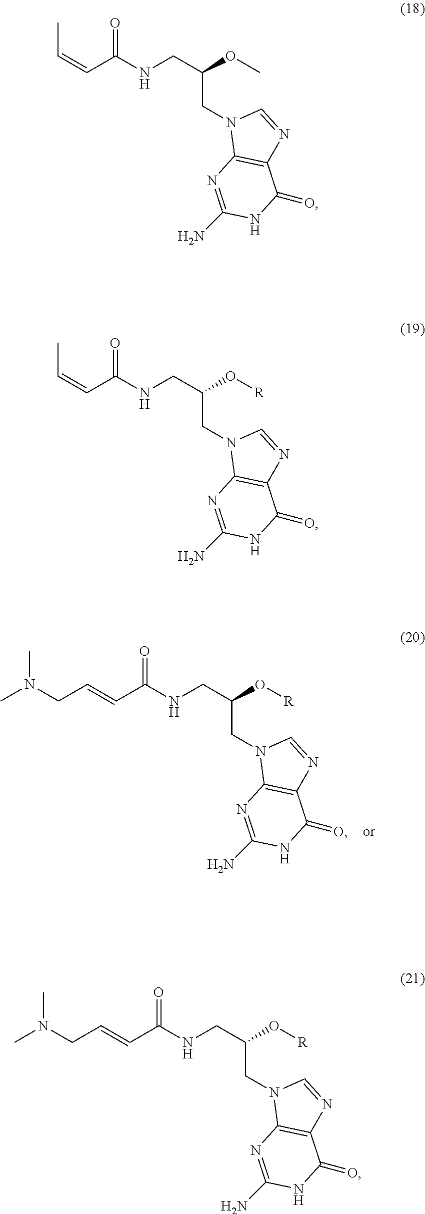

or a pharmaceutically acceptable salt thereof, wherein R is as defined anywhere herein.

In any embodiment herein, it may be that the compound described herein is a compound having the following structure:

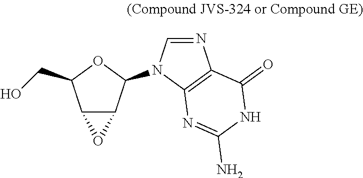 [SMILES: Nc1nc2c(ncn2[C@@H]2O[C@H](CO)[C@H]3O[C@H]32)c(=O)[nH]1]

or a pharmaceutically acceptable salt thereof.

In any embodiment herein, it may be that the compound described herein is a compound having the following structure:

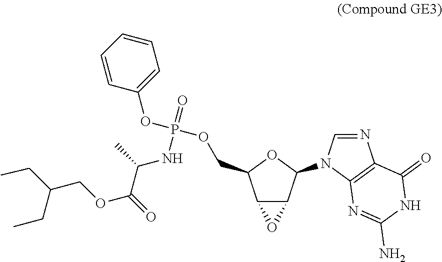 [SMILES: CCC(CC)COC(=O)[C@H](C)NP(=O)(OC[C@H]1O[C@@H](n2cnc3c(=O)[nH]c(N)nc32)[C@@H]2O[C@@H]21)Oc1ccccc1]

or a pharmaceutically acceptable salt thereof.

In any embodiment herein, it may be that the compound described herein is a compound having the following structure:

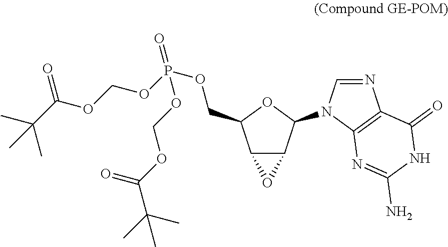 [SMILES: CC(C)(C)C(=O)OCOP(=O)(OCOC(=O)C(C)(C)C)OC[C@H]1O[C@@H](n2cnc3c(=O)[nH]c(N)nc32)[C@@H]2O[C@@H]21]

or a pharmaceutically acceptable salt thereof.

Also provided are compositions comprising, or consisting essentially thereof, or consisting of a compound or a pharmaceutical acceptable salt thereof as disclosed herein, and a carrier, such as a pharmaceutically acceptable carrier. In another aspect, provided are pharmaceutical compositions comprising a compound or a pharmaceutical acceptable salt thereof as disclosed herein, and a carrier, such as a pharmaceutically acceptable carrier.

In one aspect, pharmaceutical compositions or compositions as disclosed herein comprising, or consisting essentially thereof, or consisting of, one or more pharmaceutically acceptable excipients and/or active agents. Non-limiting examples of agents are anticancer agents that are used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

Non-limiting examples of pharmaceutically acceptable carriers” include diluents, excipients, or carriers that can be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

### Chemical Synthesis

The compounds described herein (e.g., a compound of Formula (I)-(III) such as any one of compounds (1)-(21)) can be prepared according to methods know in the art, including the exemplary syntheses provided herein, such as the synthesis shown in Schemes 1-2 and Example A.

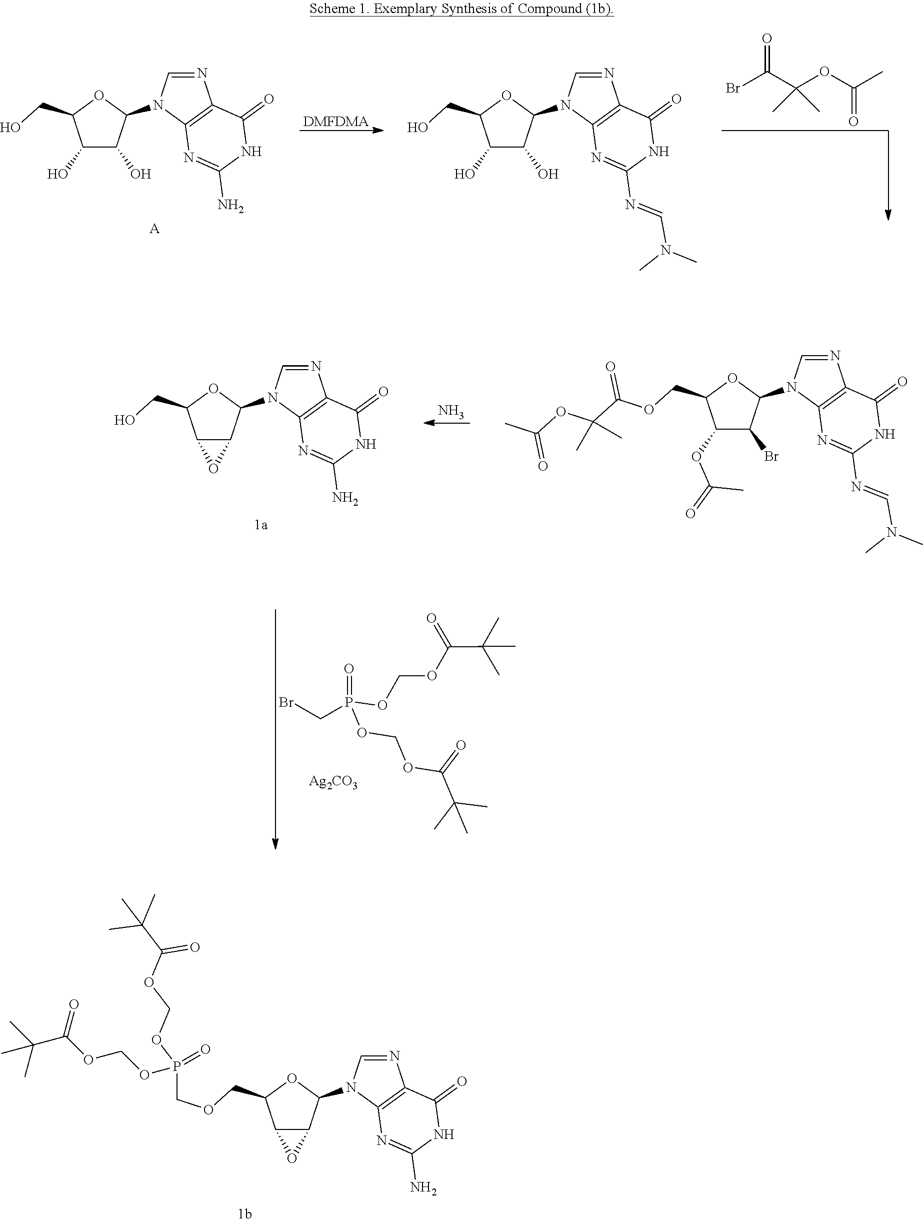

Compound (1b) can be prepared according to Scheme 1. Starting from N-protected guanosine (Compound (A)), the synthesis of guanosine-epoxide (Compound (1a)) can be achieved over three steps. Installation of the phosphorus-containing moiety to Compound (1a) furnish the synthesis of Compound (1b).

A compound of Formula (II) may be prepared using a similar method as shown in Scheme 1.

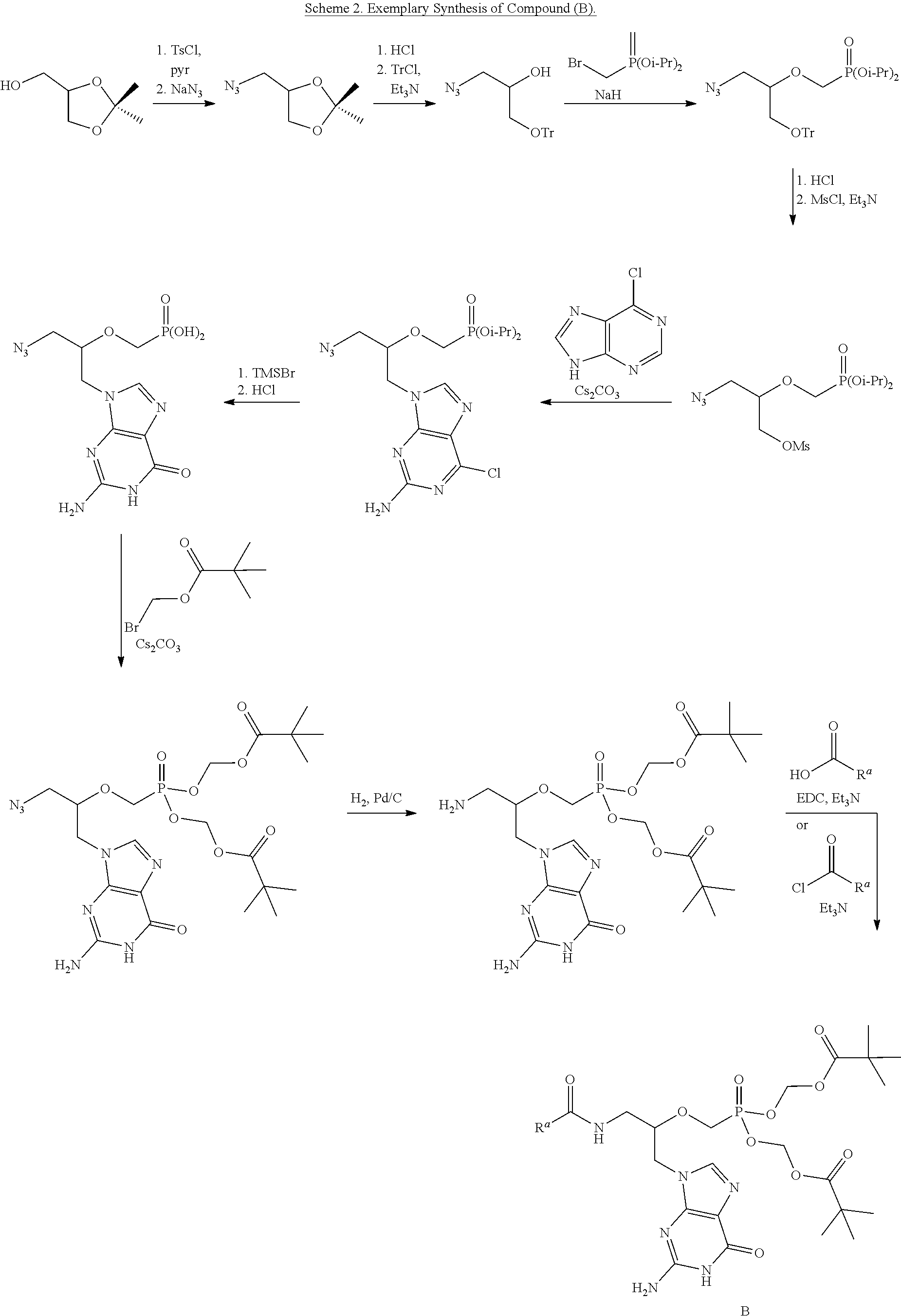

Compound (B) can be prepared according to Scheme 2. A compound of Formula (III) may be prepared using a similar method as shown in Scheme 2.

### Methods of Use

The compounds and compositions as disclosed herein are useful in methods for one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell, the method comprising, or consisting essentially of, or yet further consisting of contacting the cell with a compound or composition as disclosed herein. In one aspect the cell is a mammalian cancer cell. The contacting can be in vitro or in vivo. The cell can be from any species of cancer or tumor cell, e.g., mammalian, such as murine, simian, rat, feline, canine, bovine, ovine or human.

The methods are useful in in vitro and in vivo assays to determine effectiveness of the compound and/or composition to inhibit cell growth or modulate the pathway. The cells can be primary cells isolated from a cancer subject such as a human cancer patient or an appropriate cell line, non-limiting examples of such are provided herein.

When used in vitro, the methods can be used to identify the best therapy for the patient or subject. Thus, in one aspect, the cell can be isolated from the subject to be treated. Alternatively or in addition, they can be used to assay for effective combination therapies. When practiced in a non-human animal, the methods can be used as an animal model to assay for combination therapies or for testing personalized therapies. In one aspect, the method further comprises obtaining and implanting the subject's cells into the animal model and growing a cancer mass prior to administration of the compound or composition.

In one aspect, the cell comprises a GNAS 201 mutation. A GNAS mutation includes for example a mutation at codon 201 of the GNAS gene. In one aspect, the GNAS 201 mutation is selected from GNAS^{R201H}, GNAS^{R201C}, or GNAS^{R201C}. Methods to identify these mutations are known in the art, see, e.g., https://www.hpbonline.org/article/S1365-182X(15)30522-0/fulltext; https://www.omim.org/entry/139320; and https://pmkb.weill.cornell.edu/variants/25 (accessed on Jul. 10, 2022). Suitable subject samples are known in the art and described herein.

In another aspect, the cancer cell is selected from a cancer cell associated with appendiceal cancer, esophageal cancer, cervical cancer, peritoneal cancer, pancreatic cancer, colorectal cancer (CRC) (e.g., human colorectal cancer), colon cancer, gastric adenocarcinoma cancer, appendiceal adenocarcinoma, melanoma cancer, breast cancer, peritoneal metastasis (e.g., peritoneal metastasis in colorectal), pancreatic cystic neoplasms, fibrous dysplasia, appendiceal cancer, pancreatic cancer, intraductal papillary mucinous neoplasms (IPMN) of the pancreas, small-cell lung cancer, pancreatic cystic neoplasms, mucinous adenocarcinoma, pseudomyxoma peritonei, Signet Ring adenocarcinoma, Goblet cell mucinous adenocarcinoma, and/or tumors in lung, thyroid, bone, pituitary, stomach, intestine, colon, pancreas, or adrenocortical. In embodiments, the cancer cell is selected from an appendiceal cancer cell, an esophageal cancer cell, a cervical cancer cell, a peritoneal cancer, a pancreatic cancer cell, or colon cancer cell, optionally wherein the appendiceal cancer cell is selected from Appendiceal Adenocarcinoma (AA), Pseudomyxoma Peritone, and non-muinous AA. The cell can be a primary cancer cell isolated from a patient biopsy or a cancer cell line, and can be a primary cancer or metastatic cancer.

In a further aspect, the method further comprising contacting the cell with a second agent, prior to, concurrently with or subsequent to contacting with the compound or the composition. Non-limiting examples of agents are anticancer agents that are used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives thereof.

In a further aspect, the disease is appendiceal cancer, esophageal cancer, cervical cancer, peritoneal cancer, pancreatic cancer, colorectal cancer (CRC) (e.g., human colorectal cancer), colon cancer, gastric adenocarcinoma cancer, appendiceal adenocarcinoma, melanoma cancer, breast cancer, peritoneal metastasis (e.g., peritoneal metastasis in colorectal), pancreatic cystic neoplasms, pancreatic cystic neoplasms, fibrous dysplasia, appendiceal cancer, pancreatic cancer, intraductal papillary mucinous neoplasms (IPMN) of the pancreas, small-cell lung cancer, mucinous adenocarcinoma, pseudomyxoma peritonei, Signet Ring adenocarcinoma, Goblet cell mucinous adenocarcinoma, and/or tumors in lung, thyroid, bone, pituitary, stomach, intestine, colon, pancreas, or adrenocortical. In embodiments, the disease is mucinous appendiceal adenocarcinoma, pseudomyxoma peritonei, or non-mucinous appendiceal adenocarcinoma and/or the cancer cell is a mucinous appendiceal adenocarcinoma cancer cell, a pseudomyxoma peritonei cancer cell, or a non-mucinous appendiceal adenocarcinoma cancer cell. The disease can be a primary cancer or a metastatic cancer.

One of skill in the art will know when the contacting has been successful by assaying for cell growth inhibition or death. Thus, in one aspect, a control cell population or animal model is assayed for comparison. The control can be a positive or negative control, e.g., a companion cell assay or animal without any therapy (negative control) or one with an agent known to provide the desired benefit, e.g., inhibiting the growth of the cancer cell (positive control).

Further provided are methods of one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell in a subject in need thereof, the method comprising, or consisting essentially of, or yet further consisting of administering to the subject an effective amount of a compound or composition as disclosed herein.

Also provided is a method of treating cancer in a subject or patient in need thereof, the method comprising, or consisting essentially of, or yet further consisting of administering to the subject an effective amount of a compound or composition as disclosed herein. The cancer can be a primary cancer (localized) or metastatic, and thus selected from Stage I, Stage II, Stage III, or Stage IV. The therapy can be administered subsequent to resection of the cancer in the subject prior to or subsequent to administration.

In one aspect, the subject on whom the methods of this disclosure are carried out is a mammal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a diseased or cancer cell mediated by GNAS activity. In a further aspect, the method further comprises assaying a suitable sample from the subject for the presence of a diseased or cancer cell mediated by GNAS activity and/or a mutation in codon 201 of GNAS.

Also provided are methods for identifying a subject or patient for a method as disclosed herein by assaying for a mutation in codon 201 of GNAS. Subjects or patients having the mutation are suitable for the therapies as disclosed herein. In a further aspect, an effective amount of a compound or composition is administered to the subject or patient.

Non-limiting examples of subject to be treated are animals, mammals, simians, rabbits, bovines, ovines, equines, canines, felines and human patients. In one aspect, the subject to be treated is a human.

Any appropriate mode of administration can be used, e.g., localized or systemic. The treatment can be a first line, second line, third line or fourth line therapy.

In one aspect, the diseased cell or cancer comprises a GNAS 201 mutation. Non-limiting examples of such include a GNAS 201 mutation is selected from GNAS^{R201H}, GNAS^{R201C}, or GNAS^{R201C}.

In one aspect, the disease is mucinous appendiceal adenocarcinoma, pseudomyxoma peritonei, or non-mucinous appendiceal adenocarcinoma.

The cancer is selected from an appendiceal cancer, a peritoneal cancer, an esophageal cancer, a cervical cancer, a peritoneal cancer, a pancreatic cancer, or colon cancer, optionally wherein the appendiceal cancer is selected from Appendiceal Adenocarcinoma (AA), Pseudomyxoma Peritone, and non-muinous AA.

The methods can further comprise, or consist essentially of, or yet further consist of administering to the subject a second agent, prior to, concurrently or subsequent to contacting with the compound or the pharmaceutical composition. Non-limiting examples of second agents are described herein, e.g., an anticancer agent.

Treating a disease or cancer in a subject in need thereof refers to (1) preventing the symptoms of the disease or cancer from occurring in a subject that is predisposed or does not yet display symptoms of the disease or cancer; (2) inhibiting the disease or arresting its development; or (3) ameliorating or causing regression of the disease or cancer or the symptoms thereof. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition caused by the disease or cancer, stabilized (i.e., not worsening) state of a condition caused by the disease or cancer, delay or slowing of condition caused by the disease or cancer, progression, amelioration or palliation of condition caused by the disease or cancer, states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis or preventing the symptoms of the disease or cancer from occurring in a subject that is predisposed or does not yet display symptoms of the disease or cancer.

For the above methods, an effective amount is administered, and administration of the cell or population serves to treat the disease, attenuate any symptom or prevent additional symptoms of the preventing the symptoms of the disease or cancer from occurring in a subject that is predisposed or does not yet display symptoms of the disease or cancer. When administration is for the purposes of preventing, delaying or reducing the likelihood of cancer recurrence or metastasis, the compounds or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, intraocular, subconjunctival, sub-Tenon's, intravitreal, retrobulbar, intracameral, intratumoral, epidural and intrathecal. In some embodiments, an effective amount may be delivered or administered into a cavity formed by the resection of tumor tissue (i.e. intracavity delivery) or directly into a tumor prior to resection (i.e. intratumoral delivery). In some embodiments, administration can be intravenously, intrathecally, intraperitoneally, intramuscularly, subcutaneously, or by other suitable means of administration. The quantity and frequency of administration of each agent will be determined by such factors as the condition of the patient, and the type and severity of the patient's cancer, although appropriate dosages may be determined by clinical trials. Administration can vary with the subject and purpose of the therapy, e.g., in one aspect as an animal model to test or treat additional or combination therapies, or as a personalized model to treat a patient. Alternatively, the treatment is for veterinarian use.

The methods provided herein can be administered either alone or in combination with one or more known anti-cancer therapeutics. They may be administered as a first line therapy, a second line therapy, a third line therapy, or further therapy. Non-limiting examples of additional therapies include surgery, chemotherapy and radiation therapy. Appropriate treatment regimens will be determined by the treating physician or veterinarian.

### Kits

Also described herein is are kits comprising, or alternatively consisting essentially of, or yet further consisting of one or more of: a compound or composition, one or more optional naturally-occurring or non-naturally-occurring carrier(s), and optional instructions for use. In a further aspect, the instruction for use provide directions to conduct any of the methods disclosed herein.

The kit components, (e.g., reagents) can be packaged in a suitable container. The kit can also comprise, or alternatively consist essentially of, or yet further consist of, e.g., a carrier such as a buffering agent, a preservative or a protein-stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXPERIMENTAL EXAMPLES

The following examples are provided to illustrate and not limit the disclosure.

### Example A—Synthesis of Compounds

Compounds GE, GE3, and GE-POM were prepared according to Schemes A-B.

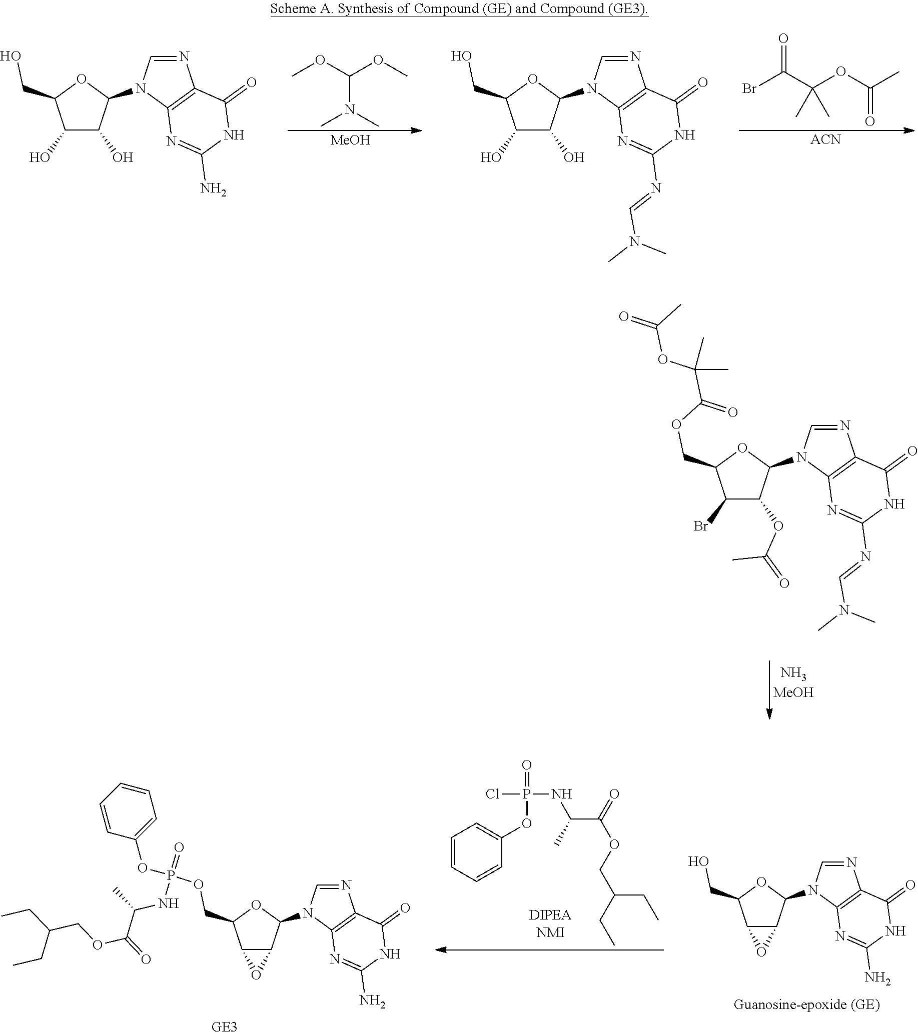

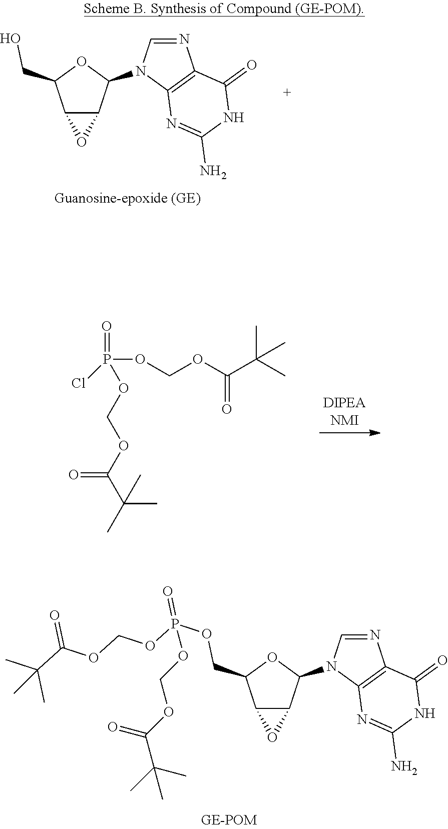 [SMILES: Nc1nc2c(ncn2[C@@H]2O[C@H](CO)[C@H]3O[C@H]32)c(=O)[nH]1 ; CC(C)(C)C(=O)OCOP(=O)(Cl)OCOC(=O)C(C)(C)C ; CC(C)(C)C(=O)OCOP(=O)(OCOC(=O)C(C)(C)C)OC[C@H]1O[C@@H](n2cnc3c(=O)[nH]c(N)nc32)[C@@H]2O[C@@H]21]

### Example 1−—Oncogene Addiction to GNAS in GNAS^{R201} Mutant Tumors

### Experimental—Materials and Methods

### Tumor Cell Lines

Human colorectal cancer (CRC) cell lines KM12, SNU175, SKCO1, and LS174T were obtained from Dr. Scott Kopetz's lab at MD Anderson Cancer Center. All cell lines were grown in RPMI-1640 medium supplemented with 10% FBS, 2 mM glutamine, and 1% Pen-Strep at standard culture conditions (37° C., 5% CO2, 95% humidity). Cells were maintained at low passages, authenticated by STR analysis and tested to rule out mycoplasma contamination.

### GNAS Knockout

KM12, SNU175 and SKCO1 were knocked out for GNAS (Transcript ID: ENST00000313949.11; Exon 5) by Synthego (Menlo Park, CA), using a guide sequence CGGGGUUGGCCAGCUCCACG. Sanger sequencing was performed to validate GNAS knockout. To obtain single clones, the cell pool was plated in 96-well plates at 0.5 cells/well and allowed to grow until a single, discrete clone could be seen in the well. Wells with single colonies were passaged and grown as standard cultures. Once enough cells were available, cells were collected to detect Gαs expression by immunoblotting as described below. The clones with the highest protein reduction were sequenced to confirm the specific mutation introduced by CRISPR editing. This process generated a GNAS^{352delG} clone from the KM12 GNAS^{KO-pool} cells.

### GNAS Overexpression

Applicant used a tet-inducible two-vector system to overexpress GNAS in LS174T cells. The first vector carried the tTS/rtTA cassette driven by CMV (pLV[Exp]-CMV>tTS/rtTA/Hygro), while the second vector carried the wild type (pLV[TetOn]-EGFP/Neo-TRE>hGNAS [NM_000516.5]/FLAG), R201C (pLV[TetOn]-EGFP/Neo-TRE>]/FLAG) or R201H (pLV[TetOn]-EGFP/Neo-TRE> [hGNAS [NM_000516.5]*(R201H)]/FLAG) GNAS under a TRE promoter. All the vectors were constructed and packaged by Vector Builder. The vector IDs are VB161122-1052kwm (first vector), VB191022-1021hcf (wild type GNAS), VB191022-1024bat (R201C mutant), and VB191022-1023nbd (R201H mutant), which can be used to retrieve detailed information about the vectors on vectorbuilder.com The expression plasmids were combined with viral packaging plasmids and transfected into HEK293T cell lines to produce virus-containing supernatant. LS174T cells were first transduced with VB161122-1052kwm and selected with hygromycin (75 μg/ml). The hygromycin-resistant cells were then transduced with the second vector and selected using G418 (800 μg/ml). Dox-induced expression of Gas (wild type or mutant) was visualized by EGFP fluorescence using the Cytation 5 imager (Biotek).

### PrestoBlue Viability Assay

Effect of GNAS knockout and overexpression on cell viability was studied using the PrestoBlue HS reagent (ThermoFisher Scientific, Waltham, USA). Cells were plated in 96-well microplates. After allowing them to adhere, PrestoBlue (10% final concentration) was added to all wells and plates were incubated at 37° C. for 1 h at standard culture conditions. Fluorescence was read at 560/590 nm using a Biotek Neo Synergy plate reader.

### Clonogenic Assay

The effect of GNAS expression on clonogenic capacity was assessed according to a previously published protocol (48). Briefly, 2000-3000 cells/well were plated in six-well plates. Cells were allowed to grow under standard culture conditions for approximately 12-15 days till colonies of at least 50 cells were visible under the microscope. The colonies were fixed in 1% paraformaldehyde and stained with 1% crystal violet. The number of colonies was counted using a Cytation Imager (Biotek).

### Immunoblot Analysis of GNAS

Expression of Gαs protein was studied by immunoblotting. Wildtype, GNAS^{KO-pool} and GNAS overexpressing cells (2×10⁶) were lysed in RIPA buffer (Cat No. 9806S, Cell Signaling Technology, MA) with protease inhibitors (Cat No. 4693124001, Sigma Aldrich, MO). Cellular proteins (60 μg) were separated by SDS polyacrylamide gel electrophoresis and electro-transferred onto a nitrocellulose membrane. The membrane was probed with monoclonal anti-GNAS antibody recognizing the full-length human recombinant GNAS protein (OT17A6, Invitrogen) followed by anti-mouse HRPO-conjugated antibody. Protein bands were detected by chemiluminescence (ECL). GAPDH expression was used as the loading control.

### Reverse Phase Protein Array Analysis

Wildtype, GNAS^{KO-pool} and GNAS overexpressing cells (5×10⁶) were lysed in buffer containing 1% Triton X-100, 50 mM HEPES, pH 7.4, 150 mM NaCl, 1.5 mM MgCl2, 1 mM EGTA, 100 mM NaF, 10 mM Na pyrophosphate, 1 mM Na3VO4, 10% glycerol, containing freshly added protease and phosphatase inhibitors (Roche Applied Science). Cell lysates (1.5 μg/μl) were submitted to the Reverse Phase Protein Array (RPPA) Core at MD Anderson for analysis.

### RNA Sequencing

Cell-line and tumor samples were submitted to Novogene Corp., Sacremento, CA_for RNA sequencing. Raw BAM/FASTQ files were mapped to the human reference genome (GRCh38) using STAR (version 2.7.2b) RNASeq alignment tool (49). Biobambam (version 0.0.191) was used to mark duplicates and index raw files before alignment (50). The Gencode (version 22) gene annotation (.gtf) file was used in the RNAseq data processing pipeline (51). HTSeq (version 0.11.0) was utilized to extract raw read counts from STAR-aligned RNAseq files (52). Xenome (version 1.0.1-r) was used to segregate mouse and human reads from raw RNAseq files of PDX models (53). Mouse reference genome (GRCm39) and gene annotation file (Gencode.vM26) were used for mapping and annotating mouse reads (https://www.ncbi.nlm.nih.gov/grc/mouse/data). Extracted human reads were processed as described above. Additionally, expression of the top GNAS encoded isoforms from the TCGA colorectal adenocarcinoma cohort (n=460) was obtained from the tool ISOexpresso (54).

### Gene Set Enrichment Analysis (GSEA)

Raw counts of the genes were normalized using the median to ratio raw counts normalization method implemented in DESeq2 (55). The normalized counts of each gene were log 2 scaled and ranked by their fold changes (FC) values between paired samples. A ranked list of genes with log 2 [FC] values was used in pre-ranked GSEA analysis (56). Hallmark gene sets of human tissues and C6: oncogenic signature gene sets available in MSigDB Collections were utilized as background gene sets database in GSEA (57). Broad institute's standalone GSEA (version 4.0.3) software was used to perform enrichment analysis (http://www.gsea-msigdb.org/gsea/index.jsp).

### GNAs Mutation Hotspots, Co-Mutation, and Zygosity Analysis

Somatic mutation profiles of 1050 GNAS mutant cancer patients from The University of Texas MD Anderson Cancer Center (UTMDACC) molecular database from 2010 to 2021 were retrospectively collected in an anonymized fashion using an institutionally approved electronic data capture resource, the Molecular and Clinical Data Integration Platform (MOCLIP). cBioPortal's web-based tool MutationMapper for plotting lollipop plots was used for identifying mutation hotspots in GNAS (58, 59) or co-mutation analysis the colorectal cancer cohort was selected as it had the most GNAS^{R201} mutations (n=122), when restricting to 2295 colorectal patients sequenced using the same CMS50 mutation panel this left 100 GNAS^{R201} mutant colorectal patients. Mutation profiles of GNAS^{R201} (n=100) and GNAS^{wt} (n=2195) CRC patients were combined to construct confusion matrices of GNAS vs other oncogenes. Fisher's exact test was performed to test significant associations between GNAS^{R201} mutation with other mutated genes in the study cohort. Two-sided p-value was calculated and Benjamini-Hochberg correction was applied to control the false positive rate of multiple hypotheses tests. False discovery rate (FDR) less than 0.05 was chosen to reject the null hypothesis. The basic Stats (version 4.0.0) library available in R statistical software (version 4.0.0) were used to perform Fisher's exact test. GNAS^{R201} mutation zygosity was determined using GNAS mutant tumor samples from cBioPortal (n=30). Relative variant allele frequency (vaf) was calculated by diving the chosen allele frequency by highest allele frequency from the data.

### Organoid Culture

KM12 and LS174T cells were suspended with Matrigel (BD) at 100 cells/μL and plated as 5 μL droplets onto 96-well multi-well culture plates. The plates were incubated at 37° C. for 15 min. After the mixture had solidified, cultures were overlaid with 100 μL of RPMI1640 medium containing 10% FBS and antibiotics. The culture medium was replaced with fresh medium every 5 days. The plates were placed in the cell imaging multi-mode plate reader Cytation™ 5 (Biotek, Winooski, VT) at 37° C. in an atmosphere supplemented with 5% CO₂. Images of each well were collected every 5 days using a 4× phase-contrast objective and a RFP filter cube (Excitation 539/40/Emission593/40, mirror 568). Two×three images and 12 slices of z-stack per well were stitched to cover most of the well. Cell images were processed using GEN5™ software (Biotek). The numbers and total area of organoids were calculated.

### Peritoneal Metastasis Model

All the animal experiments have been approved by Institutional Animal Care and Use Committee (IACUC). Female Nod/Scid/Il2rg^{null} (NSG) mice were purchased from Jackson Laboratory (Bar Harbor, Maine, USA) and housed in individually ventilated cages. Cell lines were transduced with FUW-Luc-mCherry (60) to allow non-invasive visualization of tumor growth by Lumina in vivo imaging systems (IVIS). Optimal sample size was determined by power analysis of data from pilot experiments using each cell line, using G*Power 3.1.9.7 with parameters of a err prob=0.05 and Power (1-β err prob)=0.80. There was no inclusion or exclusion criteria for mouse studies. The mice were randomly divided into 2 or more groups and injected intraperitoneally (i.p.) with the indicated numbers of luciferase-tagged CRC cell lines suspended in 500 μl of saline. For imaging, mice were administered i.p. D-luciferin (15 mg/ml in Mg/Ca-free Dulbecco's modified PBS) (Perkin Elmer, Waltham, MA, USA) and anaesthetized with 2.5% isoflurane. Light emission was recorded using Lumina IVIS (Perkin Elmer). Ten minutes after administration of luciferin, IVIS images were acquired using small binning and 1 sec exposures. Obtained data from animal studies was quantified and analyzed in a blinded fashion.

### Immunohistochemistry

For dual immunohistochemistry (IHC), sections of formalin-fixed, paraffin-embedded (FFPE) tissues (4 μm) were mounted on positively charged Superfrost Plus slides (Fisher Scientific, Pittsburgh, PA) and processed using the automated BOND-MAX system (Leica Biosystems, Buffalo Grove, IL). Following automated deparaffinization, tissue sections were subjected to automated heat-induced epitope retrieval (HIER) using a ready-to-use EDTA-based solution (pH 9.0; Leica Biosystems) for 20 min at 100° C. Subsequently, endogenous peroxidase was quenched by incubation with 3% hydrogen peroxide (5 min), followed by incubation with a recombinant rabbit anti-human b-catenin polyclonal antibody (ab16051, Abcam, Cambridge, United Kingdom) diluted at 1:1,000 in a ready-to-use antibody diluent (Dako, Agilent Technologies, Carpinteria, CA) for 30 min at room temperature. This was followed by incubation with a polymer-labeled goat anti-rabbit IgG conjugated to HRP (8 min). 3,3′-Diaminobenzidine tetrahydrochloride (DAB) was used as the substrate, and the slides were incubated for 10 min. Slides were mounted with a permanent mounting medium (Micromount; Leica Biosystems). Human tissue sections were used as positive controls, and tissue sections not incubated with primary antibodies were used as negative controls. For the quantification of β-catenin staining on the tumor sections, the intensity score derived from the average intensity of the staining of the nuclei or cytoplasm (cellular average) was determined according to the three threshold intervals (scoring 0, 1+, 2+, and 3+) set in the algorithm macro.

### Statistics

Statistical analysis was carried out using R 3.3.0 (The R Project for Statistical Computing) and GraphPad Prism 9 (GraphPad Software). Categorical analyses were carried out by ANOVA, with multiple comparisons subjected to the Dunnett test. Normalized and scaled abundances proteins and phospho-proteins were compared by using a Student's t-test. Multiple testing corrections were made using FDR according to Benjamini and Hochberg. An unpaired two-tailed Student t test was used to determine significant difference between two variables. When appropriate, Applicant have estimated variation within each group of data and ensured that it is similar between groups that are being statistically compared. The log-rank test was used to determine statistically significant differences between two Kaplan-Meier survival curves. The data analyzed meet assumptions of statistical tests performed. *, P<0.05; **, P<0.01; ***, P<0.001.

### Results

### Activating R201 Mutations in GNAS are Observed in Many Cancer Types

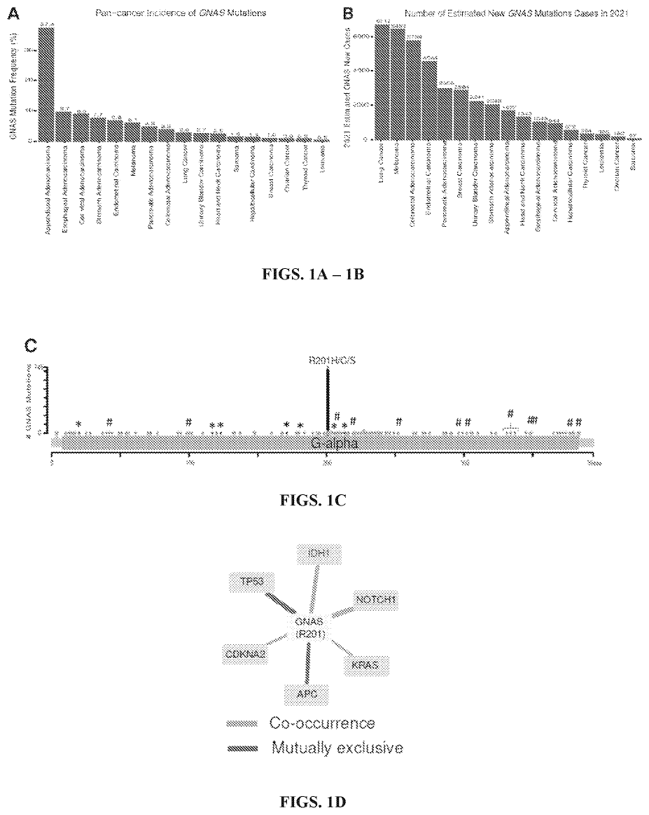

Activating mutations in the R201 codon of GNAS drive oncogenesis in various tumors including esophageal, cervical, pancreatic, and colon cancers, but they are most frequent in appendiceal adenocarcinoma (FIG. 1A). In 2021 alone, there were over 40,000 new GNAS^{R201} mutant cases in the United States, highlighting a major potential for GNAS as a therapeutic target (FIG. 1B). Analyzing a pan-cancer cohort of patients from an institutional database identified 1050 GNAS mutant tumors, the mutational pattern of these GNAS mutations showed a heavy concentration of mutations in the R201 codon, classic for a gain-of-function mutation (FIG. 1C) (12).

To minimize confounding from mixing different tumor lineages (13), co-mutations analysis was restricted to colorectal cancer patients showing that NOTCH1 (odds ratio=9.67; CI=2.68-35.09; FDR=0.0077), IDH1 (odds ratio=6.99; CI=2.44-21.00; FDR=0.0052), CDKN2A (odds ratio=3.35; CI=1.39-8.30; FDR=0.0200), and KRAS (odds ratio=3.14; CI=2.03-4.88; FDR=0.0003) genes were significantly co-mutated with GNAS^{R201}. Contrarily, loss-of-function mutations of APC (odds ratio=0.47; CI=0.30-0.75; FDR=0.0012) and TP53 (odds ratio=0.13; CI=0.08-0.22; FDR=0.0003) showed mutual exclusivity with GNAS^{R201} in the same cohort (FIG. 1D). Mutual exclusivity of GNAS and TP53 has also reported in appendiceal adenocarcinoma (14). These findings suggest that GNAS cooperates with drivers of cell proliferation but represents a different path to oncogenesis from APC-loss mediated Wnt activation or TP53-loss mediated DNA damage repair deficiency. The complete results of co-mutation analyses of GNAS^{R201} variant versus all other mutated genes are enlisted in Table 1.

TABLE 1
Co-mutation network of all GNASR201 variants with other mutant genes in colorectal
cancer samples (calculated as described in FIG. 1D) from the institutional molecular
database of The University of Texas MD Anderson Cancer Center.

| | | | | | | FDR |
|---|---|---|---|---|---|---|
| | | | | | | (Benjamini |
| | | | | | P- | and |
| | GNAS_Mutant | GNAS_Wild_Type | Odds.Ratio | Confidence.Interval | value | Hochberg) |
| APC_Mutant | 29 | 1021 | | | | |
| APC_Wild_Type | 71 | 1174 | 0.47 | 0.30-0.75 | 0.0006 | 0.0012 |
| CDKN2A_Mutant | 5 | 34 | | | | |
| CDKN2A_Wild_Type | 95 | 2161 | 3.35 | 1.39-8.30 | 0.02 | 0.02 |
| IDH1_Mutant | 4 | 13 | | | | |
| IDH1_Wild_Type | 96 | 2182 | 6.99 | 2.44-21.00 | 0.0052 | 0.0078 |
| KRAS_Mutant | 73 | 1015 | | | | |
| KRAS_Wild_Type | 27 | 1180 | 3.14 | 2.03-4.88 | 0.0001 | 3.00E−04 |
| NOTCH1_Mutant | 3 | 7 | | | | |
| NOTCH1_Wild_Type | 97 | 2188 | 9.67 | 2.68-35.09 | 0.0077 | 0.0092 |
| TP53_Mutant | 20 | 1430 | | | | |
| TP53_Wild_Type | 80 | 765 | 0.13 | 0.08-0.22 | 0.0001 | 3.00E−04 |
| RET_Mutant | 3 | 16 | | | | |
| RET_Wild_Type | 97 | 2179 | 5.55 | 1.33-17.65 | 0.0102 | 0.05266 |
| FBXW7_Mutant | 13 | 154 | | | | |
| FBXW7_Wild_Type | 87 | 2041 | 2.11 | 1.08-3.84 | 0.0178 | 0.0641 |
| AKT1_Mutant | 3 | 19 | | | | |
| AKT1_Wild_Type | 97 | 2176 | 4.44 | 1.08-13.6 | 0.0195 | 0.0641 |
| MLH1_Mutant | 4 | 20 | | | | |
| MLH1_Wild_Type | 96 | 2175 | 4.53 | 1.1-13.89 | 0.0183 | 0.0641 |
| KDR_Mutant | 3 | 18 | | | | |
| KDR_Wild_Type | 97 | 2177 | 4.93 | 1.19-15.36 | 0.0144 | 0.0641 |
| FGFR2_Mutant | 1 | 6 | | | | |
| FGFR2_Wild_Type | 99 | 2189 | 7.28 | 0.71-41.4 | 0.0459 | 0.1146 |
| ERBB2_Mutant | 4 | 27 | | | | |
| ERBB2_Wild_Type | 96 | 2168 | 3.22 | 0.81-9.48 | 0.0478 | 0.1146 |
| SMO_Mutant | 4 | 25 | | | | |
| SMO_Wild_Type | 96 | 2170 | 3.47 | 0.86-10.3 | 0.0389 | 0.1146 |
| HRAS_Mutant | 1 | 6 | | | | |
| HRAS_Wild_Type | 99 | 2189 | 7.28 | 0.71-41.4 | 0.0459 | 0.1146 |
| CDH1_Mutant | 1 | 8 | | | | |
| CDH1_Wild_Type | 99 | 2187 | 5.46 | 0.56-27.87 | 0.0696 | 0.1473 |
| CTNNB1_Mutant | 4 | 31 | | | | |
| CTNNB1_Wild_Type | 96 | 2164 | 2.82 | 0.71-8.17 | 0.0686 | 0.1473 |
| ATM_Mutant | 11 | 153 | | | | |
| ATM_Wild_Type | 89 | 2042 | 1.77 | 0.86-3.33 | 0.0773 | 0.1547 |
| FLT3_Mutant | 1 | 10 | | | | |
| FLT3_Wild_Type | 99 | 2185 | 4.37 | 0.46-20.89 | 0.0964 | 0.1826 |
| KIT_Mutant | 13 | 436 | | | | |
| KIT_Wild_Type | 87 | 1759 | 0.64 | 0.33-1.15 | 0.1593 | 0.2867 |
| ABL1_Mutant | 1 | 19 | | | | |
| ABL1_Wild_Type | 99 | 2176 | 2.17 | 0.24-9.16 | 0.2552 | 0.4176 |
| SMARCB1_Mutant | 2 | 20 | | | | |
| SMARCB1_Wild_Type | 98 | 2175 | 2.22 | 0.25-9.35 | 0.2483 | 0.4176 |
| EGFR_Mutant | 2 | 22 | | | | |
| EGFR_Wild_Type | 98 | 2173 | 2.02 | 0.23-8.39 | 0.2812 | 0.4402 |
| ERBB4_Mutant | 1 | 27 | | | | |
| ERBB4_Wild_Type | 99 | 2168 | 1.55 | 0.18-6.29 | 0.3876 | 0.5582 |
| RB1_Mutant | 1 | 28 | | | | |
| RB1_Wild_Type | 99 | 2167 | 1.55 | 0.18-6.29 | 0.3876 | 0.5582 |
| NRAS_Mutant | 2 | 93 | | | | |
| NRAS_Wild_Type | 98 | 2102 | 0.46 | 0.05-1.74 | 0.4378 | 0.6062 |
| PIK3CA_Mutant | 26 | 612 | | | | |
| PIK3CA_Wild_Type | 74 | 1583 | 0.91 | 0.55-1.46 | 0.7332 | 0.9101 |
| MET_Mutant | 7 | 211 | | | | |
| MET_Wild_Type | 93 | 1984 | 0.8 | 0.33-1.67 | 0.7296 | 0.9101 |
| STK11_Mutant | 2 | 42 | | | | |
| STK11_Wild_Type | 98 | 2153 | 1.05 | 0.12-4.13 | 0.7174 | 0.9101 |
| JAK3_Mutant | 3 | 83 | | | | |
| JAK3_Wild_Type | 97 | 2112 | 1.03 | 0.27-2.81 | 0.7955 | 0.9546 |
| BRAF_Mutant | 8 | 199 | | | | |
| BRAF_Wild_Type | 92 | 1996 | 0.87 | 0.36-1.82 | 0.8588 | 0.9849 |
| SMAD4_Mutant | 12 | 260 | | | | |
| SMAD4_Wild_Type | 88 | 1935 | 1.02 | 0.5-1.9 | 0.8755 | 0.9849 |
| FGFR3_Mutant | 1 | 42 | | | | |
| FGFR3_Wild_Type | 99 | 2153 | 1.03 | 0.12-4.04 | 1 | 1 |
| CSF1R_Mutant | 0 | 4 | | | | |
| CSF1R_Wild_Type | 100 | 2191 | 0 | 0-33.54 | 1 | 1 |
| PTEN_Mutant | 2 | 58 | | | | |
| PTEN_Wild_Type | 98 | 2137 | 0.75 | 0.09-2.92 | 1 | 1 |
| PTPN11_Mutant | 0 | 7 | | | | |
| PTPN11_Wild_Type | 100 | 2188 | 0 | 0-13 | 1 | 1 |

### GNAS^{R201} Drives Tumor Cell Growth In Vitro

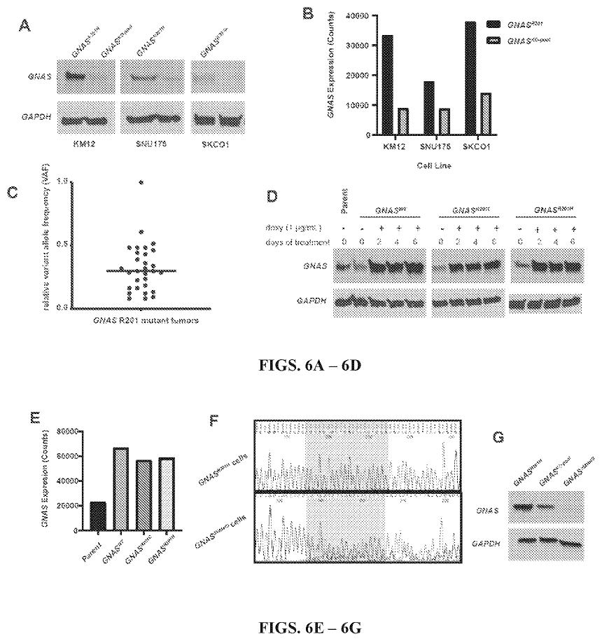

To investigate the therapeutic potential of targeting GNAS, Applicant generated isogenic GNAS-knockout and mutant overexpressing cell lines. Applicant used four human CRC cell lines with wildtype (WT) and mutant GNAS to assess the role of GNAS in the growth of peritoneal tumors. These included LS174T (GNAS^{WT}), KM12 (GNAS^{R201H}), SNU175 (GNAS^{R201C}), and SKCO1 (GNAS^{R201C}) cells (Table 2). GNAS was knocked out in KM12, SNU-175, and SKCO1 cells using ribonucleoprotein (RNP) delivery of CRISPR-Cas9. The knockout was confirmed by western blot and RNA-sequencing (FIG. 6A, FIG. 6B). Based on the Catalog of Somatic Mutations in Cancer (COSMIC) database, the R201 mutation is heterozygous in all 3 GNAS mutant cell-lines (KM12, SNU175, SKCO1). Additionally, Applicant analyzed GNAS mutant tumor samples from cBioPortal and found that the R201 mutation is almost exclusively heterozygous (FIG. 6C). Sanger sequencing and ICE analysis revealed an editing efficiency of 98% in KM12, 96% in SNU175, and 88% in SKCO1 cells. Additionally, wildtype and mutant (R201C, R201H) GNAS was overexpressed in LS174T cells using a doxycycline (doxy)-expression construct. Western blot and RNA-sequencing analyses validated significant overexpression of WT and mutant Gas (FIG. 6D, FIG. 6E).

TABLE 2
Mutational status of selected colorectal cancer cell lines.
Select Molecular Charachteristics of Colorectal Cell-Lines

| Cell Line | GNAS | KRAS | APC | TP53 | YAP1 |
|---|---|---|---|---|---|
| LS174T | WT | G12D | WT | WT | WT |
| SKCO1 | R201C | G12V | F1089fs | T491M | WT |
| SNU-175 | R201C | A59T | R232 | Q202H | WT |
| KM12 | R201H | WT | K1817fs | P72fs | WT |

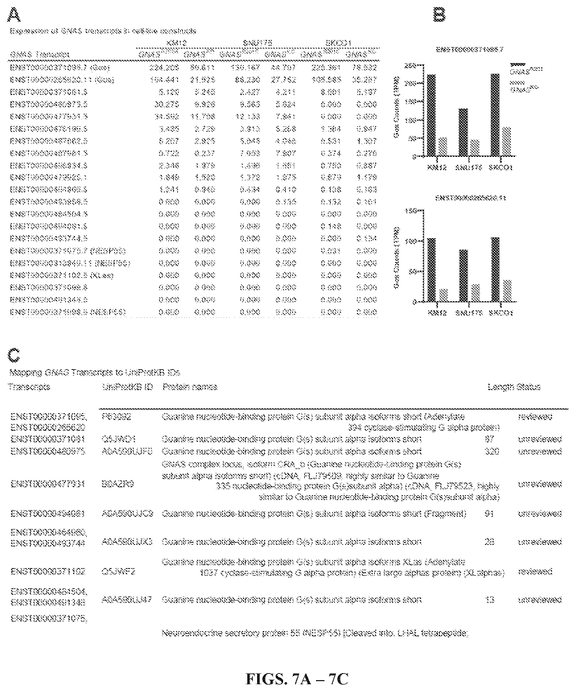

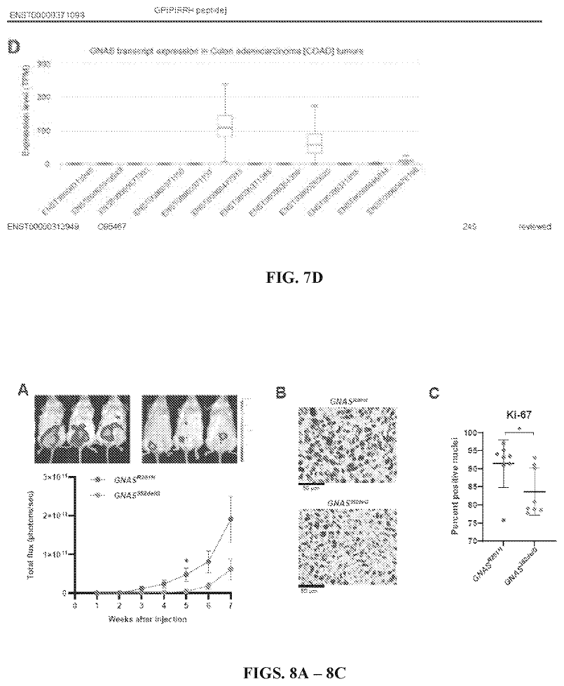

The GNAS locus encodes for multiple transcripts, including XLαs ((encoded by ENST00000371102, ENST00000371100) and NESP55 (encoded by ENST00000371075, ENST00000313949, ENST00000371098), both of which have been implicated as oncogenic in other tumor types (15) (16). In examining our RNA-seq data, Applicant saw that these alternative GNAS products were not meaningfully expressed in our cell-line models (FIG. 7A, FIG. 7C). The two GNAS transcripts that were primarily expressed (ENST00000371095, ENST00000265620) both encode the adenylate cyclase stimulating Gas (UniProtKB ID: P63092), and expression of both is reduced in GNAS knockout cell lines (FIG. 7B, FIG. 7C). Moreover, Applicant examined GNAS encoded isoform expression from a cohort of 460 colon adenocarcinoma patients from TCGA which confirmed that Gαs is the only GNAS product with meaningful expression in these tumors (FIG. 7D).

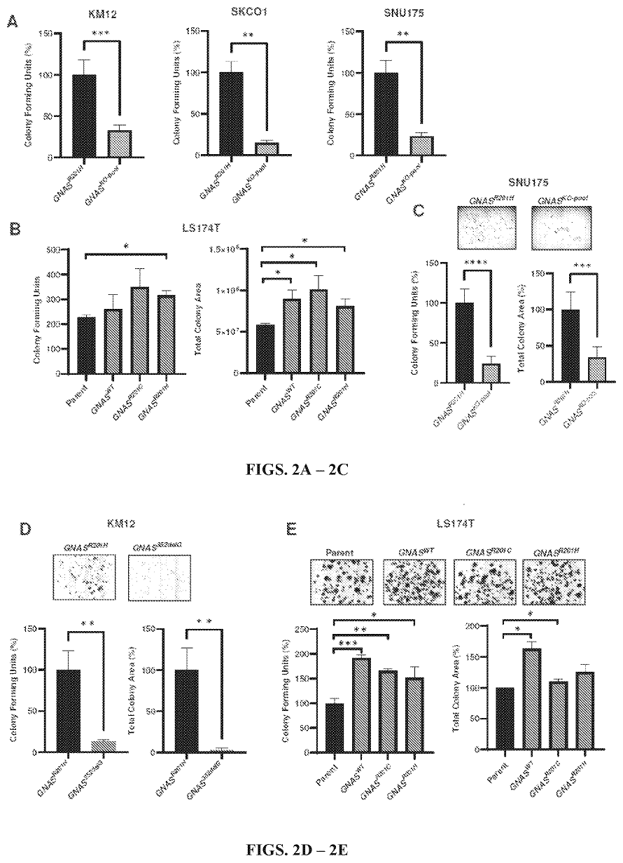

Applicant assessed the role of GNAS in cell growth in vitro by first performing colony formation assays. GNAS knockout significantly decreased 2D colony formation relative to wildtype control in KM12 (68%), SNU175 (76%), and SKCO1 (85%) cells (all p<0.0001, FIG. 2A). Similarly, Applicant observed a significant increase in colony formation following doxy-induced overexpression of GNAS^{R201C} (193%, p=0.016) and GNAS^{R201H} (170%, p=0.0037) in LS174T cells (FIG. 2B). To minimize the heterogeneous effects of the pooled GNAS knockout, Applicant generated single cells clones from the KM12 GNAS KO-pool cells. After screening many clones via western blot to confirm loss of GNAS, Sanger sequencing identified the frameshift mutation GNAS^{352delG} in one clone (FIG. 6F, FIG. 6G).

Applicant then established organoid cultures to evaluate colony formation and growth of GNAS^{352delG} KM12 cells and GNAS^{KO-pool} SNU175 cells in a three-dimensional model. Consistent with the findings in 2D culture, GNAS knockout in SNU175 (KRAS A59T) resulted in fewer (76% decrease in colony forming units, p<0.0001) and smaller (66% decrease in total colony area, p=0.0008) organoids (FIG. 2C). Similarly, for the single clone KM12 cells (KRAS WT), GNAS knockout resulted in both fewer (87.3% decrease in colony forming units, p=0.0031) and smaller (97.4% decrease in total colony area, p=0.0034) organoids (FIG. 2D). Additionally, overexpression of GNAS^{WT}, GNAS^{R201C}, and GNAS^{R201H} significantly increased growth of LS174T organoids, mirroring findings in 2D cell culture (FIG. 2E). GNAS amplification has been observed in multiple cancers including gastric adenocarcinoma (20.4%), melanoma (20.9%) and breast cancer (11.8%) supporting the idea that wildtype GNAS amplification is also oncogenic (58, 59). Taken together, these data confirm that activating mutations in GNAS promote tumor growth and suggest that GNAS mutant tumors are oncogene addicted GNAS.

### GNAS Promotes Tumor Growth In Vivo

GNAS mutation is known to be more common in peritoneal metastasis from CRC relative to primary colon tumors (19% vs 8%)²⁹, and GNAS mutation is most frequent in appendiceal adenocarcinoma (FIG. 1A) a tumor that selectively spreads to the peritoneum (8, 17). Given these findings suggesting an interaction between GNAS and the peritoneal microenvironment Applicant evaluated the growth regulatory effects of GNAS in orthotopic cell-line derived xenograft (CDX) models of peritoneal metastasis.

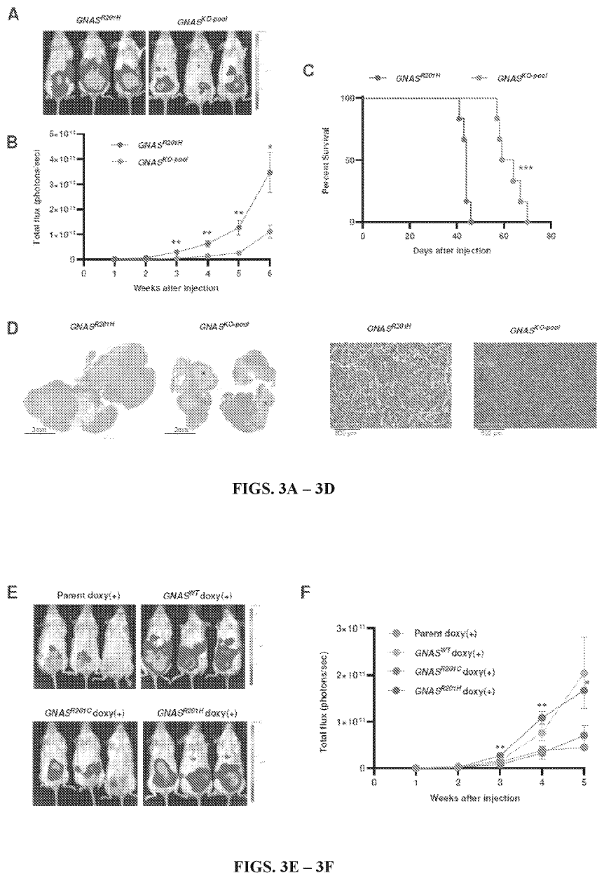

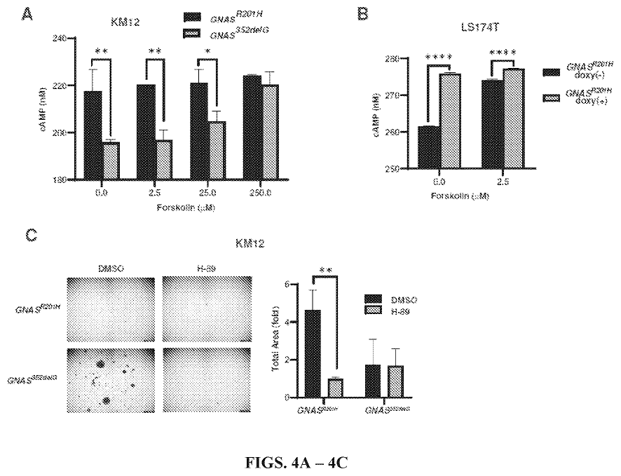

KM12 cells, which have the GNAS^{R201H} mutation, and KM12 GNAS^{KO-pool} cells were injected into the peritoneum of NSG mice. Consistent with the in vitro findings, mice injected with KM12 GNAS^{KO-pool} cells exhibited a significant reduction in tumor growth (67.9% relative to KM12 GNAS^{R201H}, p=0.016, FIG. 3A, FIG. 3B). Notably, all mice injected with KM12 GNAS^{KO-pool} cells were alive at 7 weeks whereas all mice injected with parental KM12 GNAS^{R201H} cells were dead (100% vs. 0%, p=0.0007, FIG. 3C). Histology of GNASKO-pool tumors showed a marked decrease in mucinous stroma, with cells arranged in cords, acini, and sheets, consistent with role of GNAS promoting mucin production (FIG. 3D) (18-20). The GNAS^{KO-pool} tumors also showed more lytic necrosis relative to wildtype control (FIG. 3D). The decrease in tumor growth with GNAS^{KO-pool} cells was confirmed with single clone KM12 GNAS^{352delG} cells, as expected the clonal knockout had a greater effect on tumor growth with a 77.5% decrease (n=5, p=0.07) in total flux (FIG. 8). To further confirm these findings, Applicant implanted LS174T cells overexpressing wildtype and mutant GNAS into the peritoneal space of NSG mice. Overexpression of GNAS resulted in significantly increased tumor growth [GNAS^{WT} 463% (p=0.07), GNAS^{R201C} 161% (p=0.24), and GNAS^{R201H} 379% (p=0.011) vs. Parent LS174T cells 100%, FIG. 3E, FIG. 3F].

GNAS Signals Upstream of the cAMP/PKA and Wnt/β-Catenin Pathways

In order to identify druggable downstream mediators of mutant GNAS, Applicant investigated the potential signaling pathways mediating GNAS-driven tumor growth. Gαs is known to stimulate adenylyl cyclase, leading to the accumulation of cyclic AMP (CAMP) (3). Indeed, KM12 GNAS^{352delG} cells had significantly reduced levels of cAMP relative to parental KM12 GNAS^{R201H} cells; the knockout effect could be reversed by addition of the adenylyl cyclase agonist forskolin (FIG. 4A). Similarly, in LS174T cells, overexpression of GNAS^{R201H} increased basal levels of cAMP (FIG. 4B). Forskolin increased CAMP in LS174T GNAS^{WT} cells but had no effect on the higher basal levels of CAMP in GNAS^{R201H} cells, suggesting maximal activation of adenylyl cyclase by mutant GNAS.

Without being bound by theory and to the best of Applicant's knowledge, no chemical inhibitors of Gαs exist; thus, Applicant investigated if downstream mediators of Gαs signaling could be targeted as a potential therapeutic strategy for GNAS mutant tumors. Protein Kinase A (PKA) has been reported to be activated by mutant GNAS (1, 4, 21). H-89, a potent although not completely selective PKA inhibitor (22), reduced the growth of KM12 organoids (79% vs. DMSO control, p=0.004) to a similar extent as GNAS^{352delG} (67% VS. GNAS^{R201H}, p=0.007, FIG. 4C) cells.

Interestingly, PKA inhibition did not significantly affect the growth of GNAS^{352delG} organoids, suggesting PKA is only activated with hyperactive Gαs signaling (FIG. 4C). Together these findings support PKA as a potential drug target for disrupting the effects of mutant GNAS signaling.

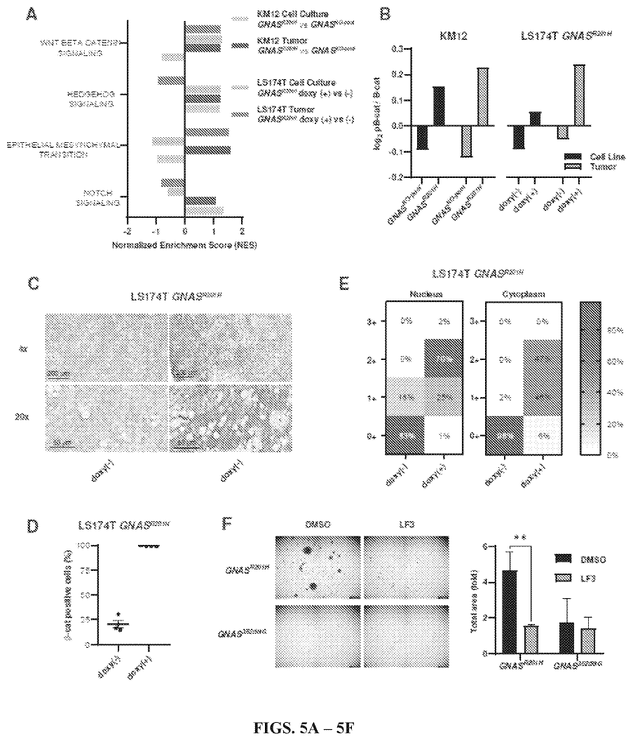

Comparing the gene expression profiles of GNAS isogenic KM12 and LS174T models, Applicant found that the Wnt Beta Catenin Signaling gene set was significantly dysregulated in 3 of 4 conditions, with greater activation of Wnt/β-Catenin in GNAS mutant samples (FIG. 5A). Moreover, reverse phase protein array (RPPA) analysis of the KM12 cells and peritoneal tumors showed increased phosphorylation of β-catenin at the Thr41/Ser45 sites in GNAS^{R201H} models relative to knockout (FIG. 5B). Previous studies demonstrated that this PKA-driven phosphorylation of β-catenin stabilizes the protein by inhibiting its ubiquitination (23), suggesting a potential pathway through which GNAS regulates activity of β-catenin. To confirm this hypothesis, Applicant stained LS174T GNAS^{R201H} overexpressing tumors with β-catenin (FIG. 5C) and observed that the percentage of nuclear β-catenin positive cells was increased from 19% to 99% (p=0.0002) by the induction of GNAS^{R201H} expression (FIG. 5D). While Applicant observed an increase in both cytoplasmic and nuclear β-catenin in GNAS^{R201H} tumors, the β-catenin stain was more intense in the nucleus following induction, with 70% nuclei having an intensity of 2+ as compared to only 47% cytoplasm having a score of 2+ (FIG. 5E). These results suggest an increased nuclear activity of β-catenin upon GNAS overexpression may drive Wnt signaling, as supported by previous literature (24, 25).

Treatment with LF3, an inhibitor that disrupts the interaction between β-catenin and its downstream nuclear transcription factor TCF4, decreased growth of KM12 GNAS^{R201H} organoids (67% vs. DMSO control, p=0.0007) but had a negligible effect on growth of GNAS^{352delG} organoids (FIG. 5F, FIG. 5G). Moreover, Applicant found that supplementation of organoid cultures with either Wnt3a or R-spondin (agonists of Wnt signaling) rescued organoid growth of GNAS-knockout cells (FIG. 9A, FIG. 9B). Thus, Applicant's findings support GNAS mediating its oncogenic effects by signaling through PKA and β-catenin.

### Discussion

Activating GNAS^{R201} mutations have been shown to induce neoplasia in many contexts, including pancreatic cystic neoplasms (6) and fibrous dysplasia (26); in these settings withdrawal of the exogenous GαsR²⁰¹ signal resulted in tumor regression. Applicant sought to determine if tumors with endogenous GNAS^{R201} mutations displayed oncogene addiction to GNAS in order to identify new targets for therapeutic intervention. GNAS knockout significantly decreased 2D colony formation in all GNAS mutant cell lines tested, and a similar result was seen in 3D organoid models. Of note, this effect maintained in both KRAS WT (KM12 cells) and KRAS mutant settings (SNU175, SKCO1 cells), which is important given the frequent co-mutation of KRAS and GNAS. These results were further validated in orthotopic xenograft models of peritoneal metastasis, confirming that GNAS^{R201} mutation tumors are addicted to oncogenic signaling mediated by GNAS. Interestingly, the degree to which GNAS^{R201} mutations promoted tumor growth in the peritoneum was markedly greater than what was seen in vitro, with up to 4-fold increase in tumor burden seen with expression of GNAS^{R201H} in wildtype cells and nearly 80% reduction in tumor burden with GNAS knockout in GNAS^{R201H} cells. This finding is consistent with prior reports linking GNAS mutation to peritoneal metastasis in colorectal (27, 28) and appendiceal (29) adenocarcinoma.

GNAS is known to promote mucin secretion through increased expression of the gel-forming mucins MUC2 and MUC5AC (18, 19). Further, GNAS mutation is associated with mucinous histology in appendiceal (8), pancreatic (30), colorectal (31), and lung tumors (32). Mucin secretion has been implicated in oncogenesis by many mechanisms including loss of epithelial cell polarity and activation of Wnt-β-catenin and NF-κB pathways and has also been reported to increase tumor cell adhesion to the peritoneum (33). Thus while the precise mechanism by which mutant Gαs signaling interacts with the peritoneal microenvironment is unknown, mucin production is thought to be an important mediator (18). Indeed, Applicant observe that parental KM12 GNAS^{R201H} tumors have a mucinous, fibrovascular stroma, while the KM12 GNAS^{352delG} tumors have less mucinous stroma and showed areas with increased lytic necrosis.

With regard to the mechanism underlying oncogene addiction to GNAS, under homeostatic conditions Gαs activates adenylate cyclase leading to the conversion of ATP to CAMP, an intracellular second messenger for several trophic hormones (34). Multiple studies have implicated that GNAS R²⁰¹ mutations constitutively activate adenylate cyclase either through decreased GTPase activity or allowing for activation in GDP-bound state ultimately causing intracellular cAMP levels to remain elevated (1, 3, 4). Applicant's studies confirm the central role of CAMP in oncogenic Gαs signaling. Downstream of CAMP Gαs signaling becomes more complex and has been noted to have significant variability based on cell lineage (1, 21). Applicant identify that at least in CRC cells, PKA is an important downstream effector of GNAS, with both chemical inhibition of PKA and genetic KO blunting the pro-growth effect of mutant GNAS. PKA has been previously implicated in the Gas pathway in adrenal tumors where gain-of-function mutations in GNAS, PKA and CTTNB1 (encoding for β-catenin) were reported as mutually exclusive (35).

PKA is also known to phosphorylate and upregulate β-catenin signaling (23, 36). Wnt has been implicated in oncogenesis in tissues carrying GNAS mutations, including thyroid (37), bone (38), pituitary (39), stomach (40), intestine (41), colon, pancreas, and adrenocortical (42) tumors. Gene expression analysis to understand the CAMP tumorigenic activity in the adrenal cortex indicated cell cycle and Wnt signaling as the most affected pathways (43). Neoplasia with GNAS mutations are typically characterized by concerted CAMP and Wnt signaling, with crosstalk potentially occurring at any level of the signaling cascade (41, 43, 44). Wnt activation leads to stabilization of cytoplasmic β-catenin, which is then translocated into the nucleus to activate transcription. Consistent with these data, Applicant find that Wnt/β-catenin is the hallmark gene set most downregulated by GNAS knockout in CRC tumors, further validated by proteomic analysis and rescue of GNAS knockout by addition of Wnt agonists.

The identification that GNAS mutant tumors are oncogene addicted to GNAS has important implications for the 40,000 patients diagnosed with GNAS mutant tumors every year in the United States. Similar to KRAS, GNAS has been difficult to drug given high-affinity for GTP, and currently no commercially available GNAS inhibitors currently exist (45). However, efforts to design cyclic peptide inhibitors are underway and this strategy is showing promise in the related G proteins GNAQ or GNA11 (46). This data suggests that an effective chemical inhibitor of Gαs would be active against GNAS mutant tumors, particularly in the peritoneal cavity. Of note, multiple inhibitors of PKA are currently in clinical development and PKA inhibition has been effective in PDX models of Gαs dependent Small Cell Lung Cancer (47). Similarly, many potential options to antagonize Wnt signaling including PORCN inhibitors, FZD antagonists, and β-catenin mediated transcription are all currently in clinical development (24). Targeting either PKA or Wnt may be effective in GNAS mutant tumors, although given the lineage specificity of G-protein signaling it should be noted our findings are derived only from CRC and may not generalize to other tumor types. Mucinous ascites represents a major source of morbidity in many peritoneal tumors, most notably low-grade appendiceal adenocarcinoma. The finding that GNAS knockout decreased mucin in peritoneal tumors suggests that Gαs inhibition could also be a palliative strategy to decrease mucinous ascites in GNAS wildtype type tumors.

### Example 2—GNAS Mutation and Tumors

### Appendiceal Tumors Are Molecularly Distinct From Colon Tumors, With Frequent GNAS Mutation

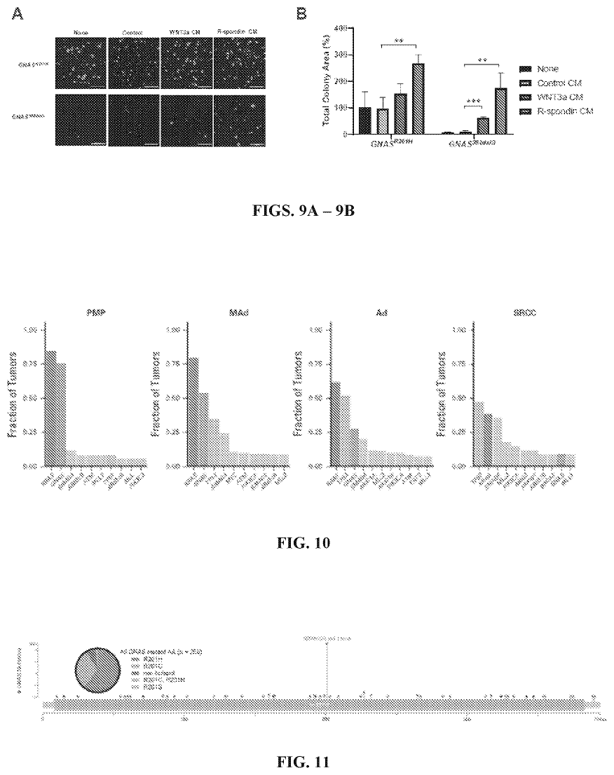

Historically, there has been an assumption of biological similarity between AA (appendiceal adenocarcinoma) and CRC due to anatomic vicinity and common embryological origin. Unlike CRC, which has a predictable spread into lymph nodes and then to the liver (70% of cases), AA rarely involves lymph nodes and even high-grade tumors almost never spread hematogenously. In general AA has a more indolent natural history relative to CRC, with median OS of 75.8 months (95% CI: 58.1-93.5) but this median disguises marked differences between high- and low-grade tumors (12). While early-stage AA can be treated definitively with surgery, there is no standard of care therapy for the systemic treatment of advanced, unresectable disease. Low-grade tumors are often managed with serial debulking surgeries combined with Heated Intraperitoneal Chemotherapy (HIPEC), however, when they progress no standard-of-care systemic treatment exists and patients often succumb to bowel obstruction or cancer cachexia from diffuse peritoneal carcinomatosis. Cohort sequencing studies by Applicant and others (1, 11, 13) have revealed APC mutation, which is a hallmark feature of CRC, is uncommon in all subtypes of AA. Frequency of GNAS mutation is much higher in AA, with a particularly strong enrichment in low grade tumors (χ² p<0.0001) (1), which also tend to show mucinous histology (FIG. 10). Unfortunately the incidence of currently ‘actionable’ mutations in genes such as HER2 or EGFR is quite rare in AA (1), as is microsatellite instability (MSI-H, less than 3% of AA cases) (1). Therefore, targeted therapy either off label or on trial is rarely an option for AA patients, making GNAS attractive as a drug target. Without being bound by theory, GNAS^{R201} tumors are oncogene addicted to Gαs signaling, and that Gαs inhibition can therefore be a successful therapeutic strategy in GNAS^{R201} tumors.

### GNAS^{R201} Tumors are Oncogene Addicted to GNAS.

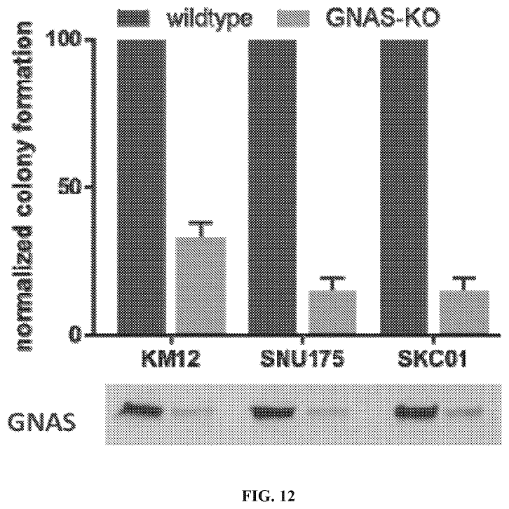

GNAS has been implicated as a driver of oncogenesis in appendiceal (13), colon (14), and gastric adenocarcinoma (15), as well as in intraductal papillary mucinous neoplasms (IPMN) of the pancreas (16) and small-cell lung cancer (SCLC) (17); GNAS^{R201} is the single most frequent cancer-causing mutation across all heterotrimeric G proteins (18). The mutational pattern of GNAS with concentration of mutations in the R²⁰¹ codon is classic for an oncogenic, gain-of-function mutation (FIG. 11) (19). Introducing activating GNAS^{R201} mutations induces neoplasia in many contexts, including pancreatic cystic neoplasms (16) and fibrous dysplasia (20); in these settings withdrawal of the exogenous GαsR²⁰¹ signal resulted in tumor regression, indicating oncogene addiction. Further, it has been shown CRISPR mediated knockout (KO) of GNAS in three CRC cell lines with GNAS^{R201} mutations (Table 3) significantly reduced colony formation in vitro (FIG. 12). The oncogenic effects GNAS was confirmed in an in vivo model of peritoneal carcinomatosis where isogenic cell lines were injected into the peritoneum of NSG mice. Given the natural history of appendix cancer is diffuse spread in the peritoneal space without hematogenous spread, this serves as an orthotopic model of metastatic appendiceal cancer. Injecting KM12 cells into the peritoneum of NSG mice readily formed tumor; tumor formation in isogenic KM12 cells with CRISPR-mediated GNAS KO was greatly reduced (FIG. 13). Similarly, over-expressing either wildtype GNAS, GNAS^{R201H}, or GNAS^{R201C} in Ls174T cells significantly increased tumor formation (data for R201C shown). In addition to profound differences in rate of tumor growth, parental KM12 tumors showed increased mucinous, fibrovascular stroma, with cells arranged in cords, acini, and sheets. The GNAS KO KM12 tumors had less mucinous stroma and showed areas with increased lytic necrosis. Interestingly, the effect of GNAS appears to be greater in the peritoneal space relative to in vitro cell culture, suggesting an interaction between GNAS and the peritoneal microenvironment. The relationship between GNAS signaling and the peritoneal microenvironment is supported by the fact that GNAS mutation occurs significantly more frequently in peritoneal metastasis relative to primary colorectal tumors (19% vs 8%, P=0.032) (21).

TABLE 3
Select molecular characteristics of cell lines

| Cell line | Tumor | GNAS | KRAS | APC | TP53 | YAP1 |
|---|---|---|---|---|---|---|
| SKCO1 | Colorectal | R201C | G12V | F1089fs | T491M | wt |
| SNU-175 | Colorectal | R201C | A59T | R232* | Q202H | wt |
| KM12 | Colorectal | R201H | wt | K1817fs | P72fs | wt |
| SNU-182 | Hepatocellular | R201S | wt | E1713E | S2151 | wt |
| LS-174T | Colorectal | wt | G12D | wt | wt | wt |

### GNAS Drives Tumor Growth Via Activation of the Wnt Pathway

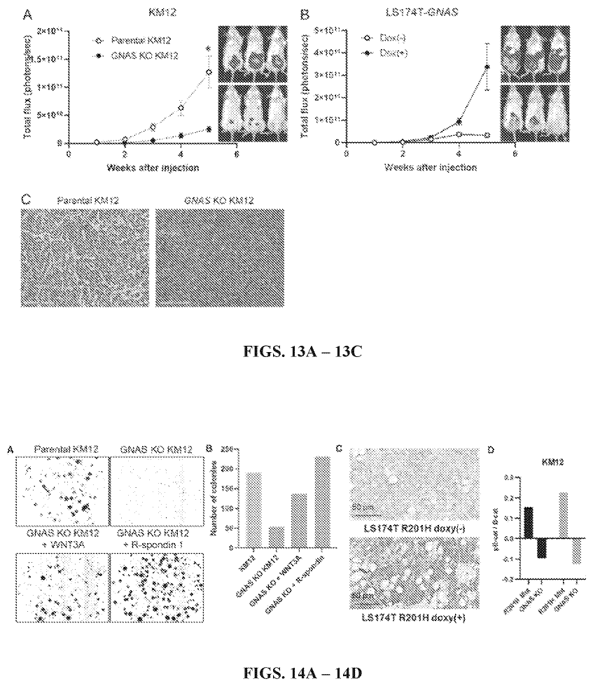

Using the same isogenic KM12 and Ls174T cell lines in 3D organoid culture Applicant found that the lethal effects of GNAS knockout can be rescued by supplementing the media with R-spondin or WNT3A. These data suggest that GNAS drives tumor growth by activating the Wnt pathway, further supported by increased nuclear β-catenin in GNAS^{R201H} and GNAS^{R201C} tumors in vivo (FIG. 14).

### Preclinical Models of Appendiceal Cancer

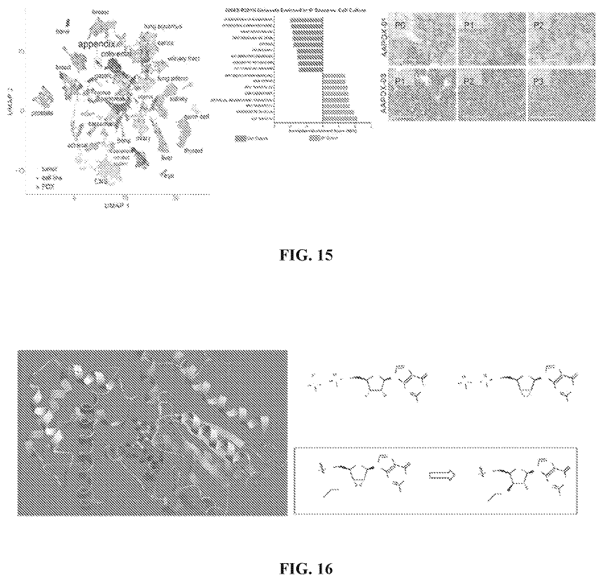

Preclinical models are critical for the drug development process, and the near complete lack of models in AA is one of the major reasons no specific therapy exists for this tumor. To rectify this problem Applicant systematically generated PDX (Patient Derived Xenograft) and PDO (Patient Derived Organoid) models of AA. To better model the unique natural history of AA, which involves early and extensive peritoneal spread but Applicant implant tumors into the peritoneal cavity. Applicant have demonstrated that the peritoneal microenvironment influences the transcriptional state of GNAS mutant tumor cells, upregulating proliferative, epithelial to mesenchymal transition (EMT), Myc, and Wnt/β-catenin gene sets (FIG. 15). Thus far six orthotopic PDX models have been created, five from MD Anderson patients and one commercially available from Jackson Lab. Profiling these tumors with RNAseq using the Celligner method (22) to perform unsupervised clustering with 29 AA tumors from MD Anderson patients and all TCGA tumors and cancer cell lines from the Cancer Cell Line Encyclopedia (CCLE) (23) Applicant find that AA tumors cluster closely together and the AA PDX clustered with human tumors. These data indicate that the orthotopic AA PDX recapitulate the transcriptional state of actual AA tumors. The histologic appearance of the AA PDX is also representative of human tumors (FIG. 15).

### Covalent Targeting of GNAS

To discover if GNAS is potentially druggable Applicant evaluated the structure of GNAS with GDP bound to identify pockets where small molecule could be developed as inhibitors. Using Molsoft ICM Pocket Finder, (24, 25), a druggable site was identified based on the x-ray crystallographic structure of GDP-bound Gαs^{R201} (26). The method is based on using experimentally determined protein structure for the prediction of druggable cavities and clefts; importantly potential substrates are not required to identify small molecule binding pockets. Key to GNAS regulation is the finding that the mutant cysteine in Gαs^{R201C} is positioned close to GDP allowing targeted covalent drugs to specifically inhibit the mutant form (FIG. 16).

### CAMP Assay to Assess GNAS Activity

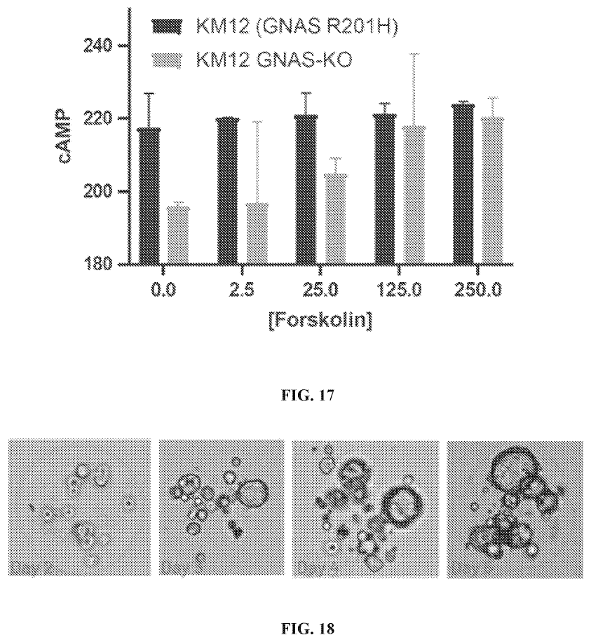

Normally heterotrimeric G proteins are active only in the GTP bound state, however it was recently shown that GαsR201 remains active even in the GDP bound state (26). Adenylyl cyclase is the immediate downstream effector of Gαs, making cellular cAMP concentration an optimal readout of Gαs inhibition. To test the efficacy of Gαs inhibitors, Applicant have optimized the bioluminescent cAMP-Glo assay (Promega) (28) using isogenic GNAS cell lines. KM12 cells which have GNAS^{R201C} show high levels of cAMP at baseline which cannot be further increased with native agonist forskolin, KM12 cells with GNAS knockout show decreased CAMP that increase in response to forskolin (FIG. 17). The CAMP-Glo assay has excellent sensitivity and has advantage that it does not require prior manipulation of cells. However, to allow for higher throughput testing in 384 well plate format the GloSensor CAMP assay (Promega) can also be optimized (29). This assay utilizes a mutant form of Photinus pyralis luciferase into which a cAMP-binding protein moiety has been inserted, Gαs activity can then be measured by monitoring change in luminescence. Both these assays are cell based, which can ensure that putative chemical inhibitors are able to permeate the cell membrane.

Without being bound by theory, Appendiceal tumors harboring the GNAS^{R201} mutation are oncogene and addicted to the constitutively active signaling of GNAS, and that these tumors can be killed by effective small molecule inhibitors of Gαs (the protein product of the gene GNAS). This hypothesis is supported by in vitro and in vivo data demonstrating that GNAS knockout is lethal to GNAS^{R201} tumors, as well as prior studies which have shown that GNAS^{R201} can drive oncogenesis in multiple tumor lineages including appendiceal, colon, and gastric cancers. Here it is our objective to design, synthesize, and characterize chemical inhibitors of Gαs.

### Example 3—Gas Inhibitors

### In Vitro, In Vivo and Pharmacokinetic Characterization of Gαs Inhibitors

Starting with the already synthesized GDP-epoxide, Gαs inhibition is measured using a cAMP assay, and in vitro viability assays are performed in GNAS^{R201} cell lines. Compounds are tested in vivo in orthotopic PDX models.

### CAMP Assay Gαs Inhibition

Using the isogenic cell lines as controls Applicant have already optimized a cAMP assay to directly measure Gαs activity. A GDP-epoxide can be first evaluated at high concentration and the diphosphate derivate at lower concentrations. Of note this assay can be performed in 384 well plate format, allowing for testing of many molecules over a broad dose range, in replicate, in one experiment.

### Testing Anti-Tumor Efficacy of Gαs Inhibitors in Cell Lines

Any compound that shows ability to inhibit Gαs in the CAMP assay can be evaluated for anti-cancer efficacy by performing in vitro cell viability assays in the KM12, SNU-175, and SKC01 cell lines. These cell lines, all derived from colorectal tumors, have naturally occurring activating GNAS^{R201} mutations. Cell growth can be measured using the using the Incucyte image-based plate reader (Sartorius), serial measurement to assess growth rate has been shown to be both more accurate and sensitive relative to end point measurements (47, 48). The Incucyte platform also allows for the simultaneous measurement of apoptosis while monitoring effects on cell proliferation. In addition to testing viability effects in the naturally GNAS mutant cell lines, the compounds to block increased proliferation when the activating GNAS^{R201} mutation is expressed in GNAS wildtype cell lines can be tested.

### Testing Anti-Tumor Efficacy of Gαs Inhibitors in PDO Models of Appendiceal Cancer

Any compound that has activity in vitro can be tested in appendix cancer specific models. Many anecdotal examples, such as the success of BRAF inhibition in BRAF V600E melanoma, but subsequent failure in BRAF V600E colon cancer, show that chemo-genetic relationships are not absolute, but are dependent on factors including cell lineage and the presence of other genomic aberrations (49). Unfortunately, appendiceal tumors, particularly low-grade tumors, are known to grow poorly in standard 2-dimensional (2D) culture conditions, a major reason why no commercially available appendix cancer cell lines exist. To overcome these limitations, a Patient-derived micro-oganospheres (PDMO) technology, can be used. This technology uses droplet-based microfluidics to partition and isolate individual cancer cells into miniaturized micro-reactors to generate microfluidic micro-oganospheres (MOS) (FIG. 18). The droplets are then patterned into high-density well plates and dosed with drug compounds; they could also be used for CRISPR screening, in vivo implantation, or other profiling experiments. It is known that immortalized 2D cell lines are not always predictive of patient outcomes (50). Patient-derived organoids (PDOs), which preserve cell-to-cell contacts important for epithelial tumors like appendix cancer, are uniquely positioned to fill this gap as, compared to cell lines, they more accurately replicate patient tumors while compared to PDXs, they improve initiation times, cost, and efficiency scales (51). Recent co-clinical trials in metastatic CRC and other gastrointestinal (GI) cancers found that PDOs successfully predicted drug response 80~90% of the time, and non-response 100% of the time, in their host patient (52-55). The main barrier to greater use of PDOs has been difficulty in generation (56, 57), especially with slow growing tumors. To circumvent these barriers, Applicant have leveraged recent technological advances in emulsion microfluidics and droplet generators to develop PDMOs, a miniaturized version of organoids that can be established rapidly and efficiently.

### Testing Pharmacokinetics and Anti-Tumor Efficacy of Gαs Inhibitors In Vivo

Fresh tumors can be harvested and 0.5 cm³ tumor blocks mixed with Matrigel and (1) planted directly into the peritoneal cavity of SCID mice by means of a 1 cm transverse abdominal incision performed under aseptic conditions; (2) implanted into the flank of a 2^{nd} SCID mouse. Thus far nine patient tumors have been implanted in addition to the three from outside sources (Table 4). These experiments have recapitulated the more aggressive nature of high-grade tumors (5 of 6 tumors formed tumor in at least one mouse) relative to low-grade (zero of 4), and also the importance of the interaction between tumor cells and the peritoneal microenvironment (tumor formation in 5 of 6 in peritoneum vs. 1 of 6 in flank for high-grade tumors).

TABLE 4
List of PDX model implanted.

| MDA-ID | PDX ID | Age | Sex | Histology | Grade | Microsatellite | GNAS |
|---|---|---|---|---|---|---|---|
| AAPDX-05 | MAd20-002b | 64 | F | Mucinous | G1 | MSS | R201H |
| | | | | adenocarcinoma | | | |
| AAPDX-06 | MAd20-003 | 61 | F | Mucinous | G1 | MSS | wild type |
| | | | | adenocarcinoma | | | |
| AAPDX-07 | MAd20-004 | 68 | F | Mucinous | G1 | MSS | wild type |
| | | | | adenocarcinoma | | | |
| AAPDX-08 | MAd20-005 | 33 | F | Mucinous | G1 | MSS | wild type |
| | | | | adenocarcinoma | | | |
| AAPDX-12 | MAd21-007 | 28 | M | Mucinous | G1 | MSS | wild type |
| | | | | adenocarcinoma | | | |
| AAPDX-03 | MAd20-001 | 61 | F | Mucinous | G2 | MSI-H | wild type |
| | | | | adenocarcinoma | | | |
| AAPDX-04 | MAd20-002a | 64 | F | Mucinous | G2 | MSS | wild type |
| | | | | adenocarcinoma | | | |
| AAPDX-10 | PMP-2 | 56 | F | Pseudomyxoma | G2 | MSS | R201C |
| | | | | peritonei | | | |
| AAPDX-01 | TM00351 | 49 | F | Mucinous | G3 | MSS | wild type |
| | | | | adenocarcinoma | | | |
| AAPDX-09 | MAd21-006 | 64 | M | Mucinous | G3 | MSS | R201H |
| | | | | adenocarcinoma | | | |
| AAPDX-11 | PMCA-3 | 62 | M | Signet Ring | G3 | MSS | R201C |
| | | | | adenocarcinoma | | | |
| AAPDX-13 | MAd21-008 | 42 | F | Goblet cell mucinous | G3 | MSS | wild type |
| | | | | adenocarcinoma | | | |

| MDA-ID | PDX ID | KRAS | BRAF | TP53 | Flank | Peritoneum |
|---|---|---|---|---|---|---|
| AAPDX-05 | MAd20-002b | G12D | wild type | wild type | 0/2 (0%) | 0/3 (0%) |
| AAPDX-06 | MAd20-003 | wild type | wild type | wild type | 0/2 (0%) | 0/3 (0%) |
| AAPDX-07 | MAd20-004 | wild type | wild type | D281Y, | 0/2 (0%) | 0/3 (0%) |
| | | | | C176F | | |
| AAPDX-08 | MAd20-005 | wild type | wild type | wild type | 0/2 (0%) | 0/3 (0%) |
| AAPDX-12 | MAd21-007 | G12D | wild type | wild type | pending | pending |
| AAPDX-03 | MAd20-001 | wild type | V600E | wild type | 0/6 (0%) | 8/9 (89%) |
| AAPDX-04 | MAd20-002a | G13V | wild type | wild type | 0/2 (0%) | 0/3 (0%) |
| AAPDX-10 | PMP-2 | G12V | wild type | wild type | pending | 4/5 (80%) |
| AAPDX-01 | TM00351 | G12D | wild type | P72R, | 8/8 (100%) | 13/13 (100%) |
| | | | | V274F | | |
| AAPDX-09 | MAd21-006 | wild type | wild type | wild type | 0/1 (0%) | 2/5 (40%) |
| AAPDX-11 | PMCA-3 | wild type | V600E | wild type | pending | 3/5 (60%) |
| AAPDX-13 | MAd21-008 | wild type | wild type | wild type | pending | pending |

Right most columns show the take rate of PDX (number of mice with tumor/number of mice implanted) at either flank or peritoneum. Mice implanted less than 90 days ago listed as pending. Note that none of the lowrgrade (G1) tumors grew in mice, for intermediate (G2) or high (G3) tumors the take rate was significantly better in the peritoneum relative to flank.

The pharmacokinetics of compounds is established. For example, RNAseq analysis of three post-treatment samples from a trial of the MEK inhibitor cobimetinib with atezolizumab in appendiceal cancer (NCT03108131) indicated that MEK regulated genes were not down regulated, suggesting that there was not adequate drug concentration in the peritoneal space. Blood samples from the mice are drawn after drug administration to determine important pharmacokinetic parameters such as oral vs. intravenous bioavailability and half-life. Since appendiceal tumors spread within the peritoneal cavity, ascites fluid can be sampled to check drug accumulation there. As a final step to ensure adequate dosing, tumors are extracted post-treatment and RNAseq is performed to observe expression changes similar to genetic GNAS knockout positive controls. As an alternate strategy Applicant can administer the drugs directly to the peritoneal space. Without being bound by theory, IP administration may be an attractive option in appendiceal cancer given that these tumors almost never extend beyond the peritoneum, and most complications from AA, such as bowel obstruction, are a result of peritoneal disease.

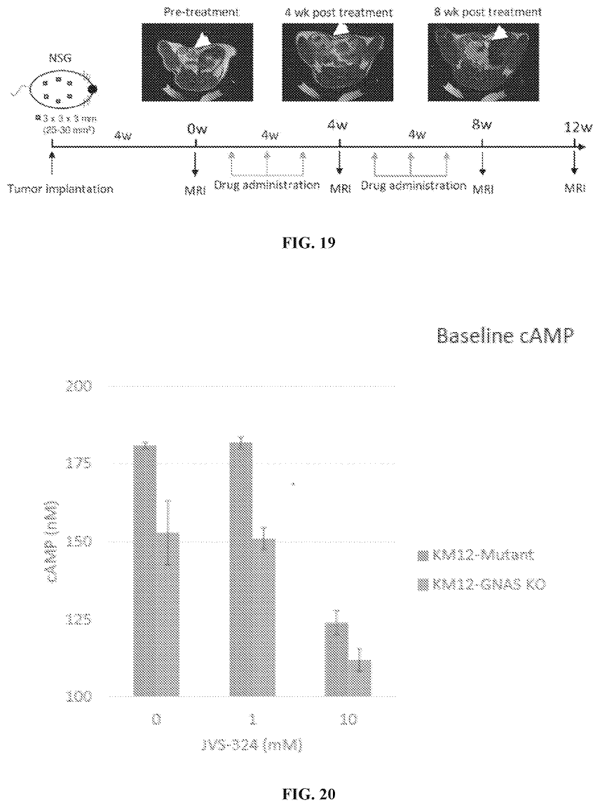

To measure the efficacy of Gαs inhibitors treating appendiceal tumor in vivo a protocol of implanting a fixed tumor volume is used allowing the tumor to establish to recreate peritoneal metastasis from appendix cancer, then treating with drug and monitoring response with serial MRI (FIG. 19). Applicant has established that four mice per experimental group gives adequate power to observe with statistical significance a 20% reduction in tumor volume. As a control for specific compounds can be tested in both GNAS mutant and wildtype PDX. Covalent inhibitors of GNAS^{R201C} should have no effect on wildtype PDX as without the cysteine no covalent bond can be formed. Non-covalent inhibitors may show some inhibition of tumor growth, or decreased production of mucinous ascites in wildtype PDX given the known role of GNAS in promoting mucin secretion through expression of MUC2 and MUC5A C (60, 61).

### Example 4—Gas Inhibitor—Compound JVS-324

A study was conducted to examine the effect of compound JVS-324 as a Gαs Inhibitor. Cells (10,000/well/384 well plate) were treated with JVS-324 (1 and 10 mM) for 20 hours. cAMP levels were measured using the CAMP-Glo™ Assay (Promega). The following results were observed: 1) cAMP levels were lower in the GNAS-KO cells; 2) JVS 324 at 1 mM had no effect on the CAMP levels; and 3) JVS 324 at 10 mM significantly reduced cAMP levels in both mutant and GNAS-KO KM12 cells, but more in GNAS-KO KM12 cells (FIG. 20).

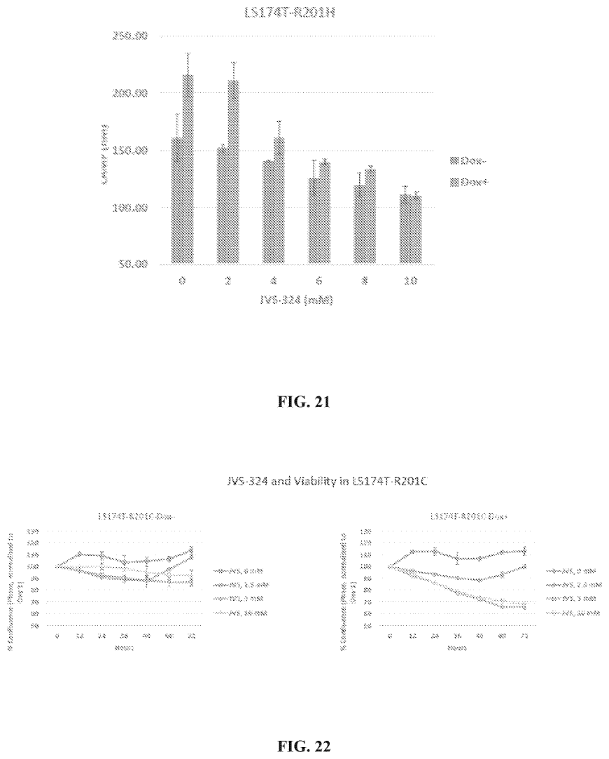

The Ls174T cell line was transduced with lentivirus to contain a construct for doxycycline inducible expression of GNAS^{R201C}, this model system has been previously described (Oncogene. 2022 August; 41 (35):4159-4168). cAMP levels were measured using the CAMP-Glo™ Assay (Promega). JVS-324 was added in increasing concentrations and cells were exposed to drug for 20 hours. The following results were observed: 1) adding doxycycline and expressing GNAS^{R201C} increased cAMP levels; 2) JVS-324 decreased the amount of CAMP in a dose dependent fashion, with greater effect on the GNAS^{R201C} cells relative to control; and 3) at 10 mM dose of JVS-324, the level of CAMP was the same between GNAS^{R201C} and control cells inhibits. Additionally, it was found that these results were consistent with JVS-324 inhibiting the function of GNAS^{R201C}. Without being bound by theory, the fact that at 10 mM dose the level of CAMP is the same between GNAS^{R201C} and control suggests that maximal inhibition of the mutant has been achieved at that dose (FIG. 21).

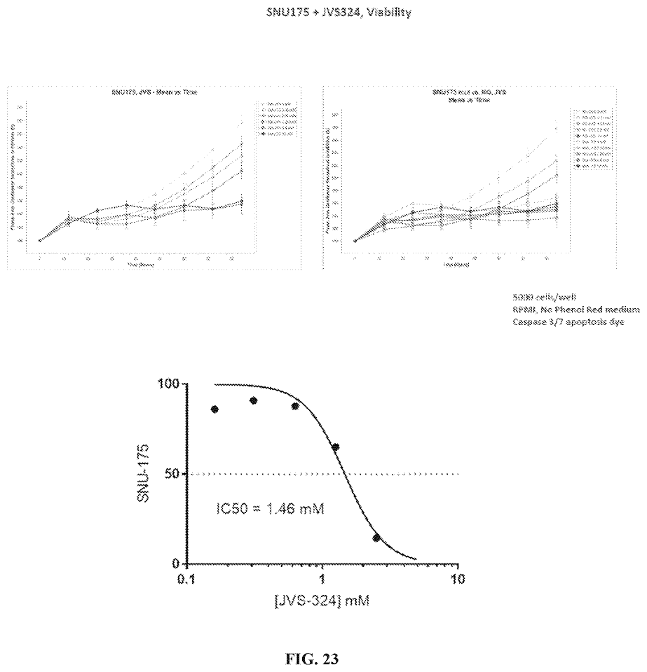

The Ls174T cell line was transduced with lentivirus to contain a construct for doxycycline inducible expression of GNAS^{R201C}. Viability was measured over time but determining cell confluence in experimental well using Incucyte Live-Cell Analysis system (Sartorius). JVS-324 was added in increasing concentrations and cell growth monitored for 72 hours. Confluency evaluation was normalized time point zero. The following results were observed: 1) JVS-324 had a dose dependent effect on viability of Ls174T cells expressing GNAS^{R201C} with toxicity seen as low as 2.5 mM; and 2) the toxicity of JVS-324 was less on Ls174T cells not expressing GNAS^{R201C}, consistent with JVS-324 showing selectivity for the mutant GNAS (GαS protein) (FIG. 22).

Viability was measured over time but determining cell confluence in experimental well using Incucyte Live-Cell Analysis system (Sartorius). JVS-324 was added in increasing concentrations and cell growth monitored for 72 hours. Confluency evaluation was normalized time point zero. An isogenic paired cell line was created by using CRISPR-Cas9 to knockout (KO) GNAS from the SNU-175 cell line; this model system has been previously described (Oncogene. 2022 August; 41 (35):4159-4168). The following results were observed: 1) JVS-324 had a dose dependent effect on viability of SNU-175 cells (which have GNAS^{R201C}) toxicity seen starting at 0.16 mM (160 μM) dose, growth arrest was nearly complete at 2.5 mM and 10 mM doses (see left panel, this same data is plotted again in R panel to contrast the KO); 2) the GNAS KO SNU-175 cells had significantly slower growth relative to SNU-175 (on the right panel), while the addition of JVS-324 did not appear to further decrease viability in the GNAS KO SNU-175; 3) the IC₅₀ for JVS-324 in SNU-175 cells was 1.46 mM; and 4) these results were consistent with JVS-324 killing SNU-175 by inhibiting GNAS^{R201C} (FIG. 23).

### Example 5—Gαs Inhibitor—Compound GE3

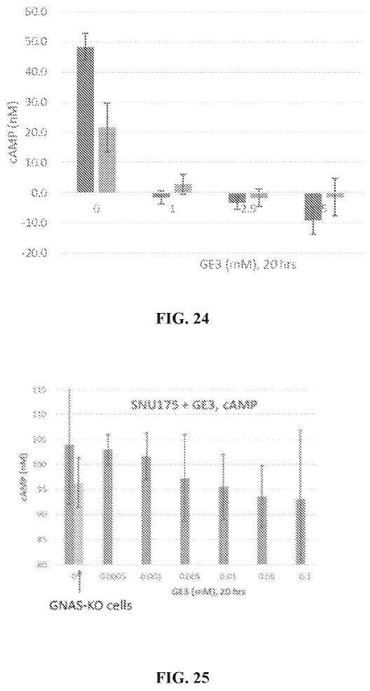

A study was conducted to examine the effect of compound GE3 as a Gαs Inhibitor. Cells were treated with compound GE3 at high (1-5 mM) and low (0-0.5 mM) doses for 20 hours. cAMP levels were measured using the CAMP-Glo™ Assay (Promega). An isogenic paired cell line was created by using CRISPR-Cas9 to knockout (KO) GNAS from the SNU-175 cell line; this model system has been previously described (Oncogene. 2022 August; 41(35):4159-4168). The following results were observed: 1) cAMP levels were lower in the GNAS-KO cells; and 2) GE3 at mM doses significantly reduced CAMP in both SNU-175 and GNAS KO isogenic paired cell line (FIG. 24).

The same experimental system was used as above to test lower doses of GE3. The following results were observed: 1) cAMP levels were lower in the GNAS-KO cells; GE3 reduced cAMP in both SNU-175 cells in dose-dependent fashion; 2) by 0.01 mM (10 uM) dose the CAMP level in the SNU-175 cells was comparable to the level in the KO cells, suggesting complete inhibition at that dose; and 3) GE3 was significantly more potent than JVS-324 (FIG. 25).

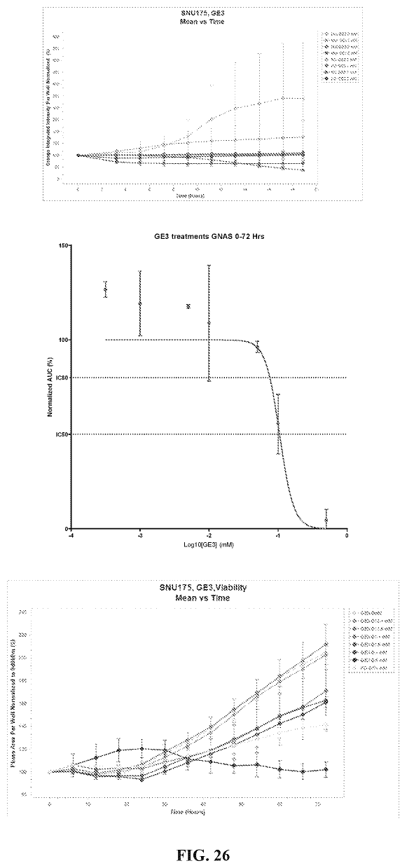

Viability was measured over time but determining cell confluence in experimental well using Incucyte Live-Cell Analysis system (Sartorius); two experiments performed. The following results were observed: 1) GE3 at mM doses significantly reduced growth of both SNU-175 and GNAS KO isogenic paired cell line, with essentially no growth seen by 1 mM; 2) growth rate was slower in the GNAS-KO cells relative to parental SNU-175; 3) GE3 reduced cell growth in SNU-175 cells in dose-dependent fashion; by 0.01 mM (10 uM) dose the growth of SNU-175 cells was comparable to the level in the KO cells, suggesting complete inhibition at that dose; and 4) the IC₅₀ of GE3 in this viability assay was 105 uM. (FIG. 26).

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES FOR EXAMPLE 1

1. O'Hayre M, Degese M S, Gutkind J S. Novel insights into G protein and G protein-coupled receptor signaling in cancer. Current opinion in cell biology. 2014; 27:126-35.
2. Rosenbaum D M, Rasmussen S G F, Kobilka B K. The structure and function of G-protein-coupled receptors. Nature. 2009; 459(7245):356-63.
3. Landis C A, Masters S B, Spada A, Pace A M, Bourne H R, Vallar L. GTPase inhibiting mutations activate the alpha chain of Gs and stimulate adenylyl cyclase in human pituitary tumours. Nature. 1989; 340(6236):692-6.
4. Hu Q, Shokat K M. Disease-Causing Mutations in the G Protein Gαs Subvert the Roles of GDP and GTP. Cell. 2018; 173(5):1254-64.e11.
5. Weinstein L S, Liu J, Sakamoto A, Xie T, Chen M. Minireview: GNAS: normal and abnormal functions. Endocrinology. 2004; 145(12):5459-64.
6. Ideno N, Yamaguchi H, Ghosh B, Gupta S, Okumura T, Steffen D J, et al. GNASR201C Induces Pancreatic Cystic Neoplasms in Mice That Express Activated KRAS by Inhibiting YAP1 Signaling. Gαstroenterology. 2018; 155(5):1593-607.e12.
7. Patra K C, Kato Y, Mizukami Y, Widholz S, Boukhali M, Revenco I, et al. Mutant GNAS drives pancreatic tumourigenesis by inducing PKA-mediated SIK suppression and reprogramming lipid metabolism. Nature Cell Biology. 2018; 20(7):811-22.
8. Ang CSP, Shen J P, Hardy-Abeloos C J, Huang J K, Ross J S, Miller V A, et al. Genomic Landscape of Appendiceal Neoplasms. JCO Precision Oncology. 2018(2):1-18.
9. Sassone-Corsi P. The Cyclic AMP Pathway. Cold Spring Harbor Perspectives in Biology. 2012; 4(12): a011148.
10. Rosciglione S, Thériault C, Boily M-O, Paquette M, Lavoie C. Gαs regulates the post-endocytic sorting of G protein-coupled receptors. Nature communications. 2014; 5:4556.
11. Schatoff E M, Leach B I, Dow L E. Wnt Signaling and Colorectal Cancer. Current Colorectal Cancer Reports. 2017; 13(2):101-10.
12. Vogelstein B, Papadopoulos N, Velculescu V E, Zhou S, Diaz L A, Kinzler K W. Cancer genome landscapes. science. 2013; 339(6127):1546-58.
13. Wang S, Ma J, Zhang W, Shen J P, Huang J, Peng J, et al. Typing tumors using pathways selected by somatic evolution. Nature Communications. 2018; 9(1):4159.
14. Ang C S-P, Shen J P, Hardy-Abeloos C J, Huang J K, Ross J S, Miller V A, et al. Genomic Landscape of Appendiceal Neoplasms. JCO precision oncology. 2018; 2:PO.17.00302.
15. Garcia-Murillas I, Sharpe R, Pearson A, Campbell J, Natrajan R, Ashworth A, et al. An siRNA screen identifies the GNAS locus as a driver in 20q amplified breast cancer. Oncogene. 2014; 33(19):2478-86.
16. Faias S, Duarte M, Pereira L, Chaves P, Cravo M, Dias Pereira A, et al. Methylation changes at the GNAS imprinted locus in pancreatic cystic neoplasms are important for the diagnosis of malignant cysts. World J Gastrointest Oncol. 2020; 12(9):1056-64.
17. Raghav K, Shen J P, Jácome A A, Guerra J L, Scally C P, Taggart M W, et al. Integrated clinico-molecular profiling of appendiceal adenocarcinoma reveals a unique grade-driven entity distinct from colorectal cancer. British Journal of Cancer. 2020; 123(8):1262-70.
18. Lin Y-L, Ma R, Li Y. The biological basis and function of GNAS mutation in pseudomyxoma peritonei: a review. Journal of Cancer Research and Clinical Oncology. 2020; 146(9):2179-88.
19. Komatsu H, Tanji E, Sakata N, Aoki T, Motoi F, Naitoh T, et al. A GNAS Mutation Found in Pancreatic Intraductal Papillary Mucinous Neoplasms Induces Drastic Alterations of Gene Expression Profiles with Upregulation of Mucin Genes. PloS one. 2014; 9: e87875.
20. Nishikawa G, Sekine S, Ogawa R, Matsubara A, Mori T, Taniguchi H, et al. Frequent GNAS mutations in low-grade appendiceal mucinous neoplasms. British journal of cancer. 2013; 108(4):951-8.
21. Arang N, Gutkind J S. G Protein-Coupled receptors and heterotrimeric G proteins as cancer drivers. FEBS letters. 2020; 594(24):4201-32.
22. Lochner A, Moolman J A. The many faces of H89: a review. Cardiovascular drug reviews. 2006; 24(3-4):261-74.
23. Taurin S, Sandbo N, Qin Y, Browning D, Dulin N O. Phosphorylation of β-Catenin by Cyclic AMP-dependent Protein Kinase. Journal of Biological Chemistry. 2006; 281(15):9971-6.

24 Jung Y-S, Park J-I. Wnt signaling in cancer: therapeutic targeting of Wnt signaling beyond β-catenin and the destruction complex. Experimental & Molecular Medicine. 2020; 52(2):183-91.
25. Parsons M J, Tammela T, Dow L E. WNT as a Driver and Dependency in Cancer. Cancer discovery. 2021; 11(10):2413-29.
26. Khan S K, Yadav P S, Elliott G, Hu D Z, Xu R, Yang Y. Induced GnasR201H expression from the endogenous Gnas locus causes fibrous dysplasia by up-regulating Wnt/β-catenin signaling. Proceedings of the National Academy of Sciences of the United States of America. 2018; 115(3): E418-E27.
27. Stein M K, Williard F W, Xiu J, Tsao M W, Martin M G, Deschner B W, et al. Comprehensive tumor profiling reveals unique molecular differences between peritoneal metastases and primary colorectal adenocarcinoma. Journal of Surgical Oncology. 2020; 121(8):1320-8.
28. Stein M K, Williard F W, Tsao M, Deschner B, Dickson P V, Glazer E S, et al. Molecular comparison of primary colorectal cancer (pCRC) with peritoneal metastases (PM). Journal of Clinical Oncology. 2019; 37(4_suppl):498-.
29. Patel A, Cushman-Vokoun A M, Alex B, Sleightholm R, Foster J M. Mutations in GI cancers with peritoneal metastasis. Journal of Clinical Oncology. 2015; 33(15_suppl): e22030-e.
30. Hosoda W, Sasaki E, Murakami Y, Yamao K, Shimizu Y, Yatabe Y. GNAS mutation is a frequent event in pancreatic intraductal papillary mucinous neoplasms and associated adenocarcinomas. Virchows Archiv: an international journal of pathology. 2015; 466(6):665-74.
31. Khan M, Loree J M, Advani S M, Ning J, Li W, Pereira AAL, et al. Prognostic Implications of Mucinous Differentiation in Metastatic Colorectal Carcinoma Can Be Explained by Distinct Molecular and Clinicopathologic Characteristics. Clinical colorectal cancer. 2018; 17(4): e699-e709.
32. Ritterhouse L L, Vivero M, Mino-Kenudson M, Sholl L M, Iafrate A J, Nardi V, et al. GNAS mutations in primary mucinous and non-mucinous lung adenocarcinomas. Modern Pathology. 2017; 30(12):1720-7.
33. Kufe D W. Mucins in cancer: function, prognosis and therapy. Nature reviews Cancer. 2009; 9(12):874-85.
34. Zhang H, Kong Q, Wang J, Jiang Y, Hua H. Complex roles of CAMP-PKA-CREB signaling in cancer. Experimental Hematology & Oncology. 2020; 9(1):32.
35. Goh G, Scholl U I, Healy J M, Choi M, Prasad M L, Nelson-Williams C, et al. Recurrent activating mutation in PRKACA in cortisol-producing adrenal tumors. Nature Genetics. 2014; 46(6):613-7.

36 Hino S-i, Tanji C, Nakayama K I, Kikuchi A. Phosphorylation of β-Catenin by Cyclic AMP-Dependent Protein Kinase Stabilizes β-Catenin through Inhibition of Its Ubiquitination. Molecular and Cellular Biology. 2005; 25(20):9063-72.
37. Sastre-Perona A, Santisteban P. Role of the Wnt Pathway in Thyroid Cancer. Frontiers in Endocrinology. 2012; 3.
38. Regard J B, Cherman N, Palmer D, Kuznetsov S A, Celi F S, Guettier J-M, et al. Wnt/-catenin signaling is differentially regulated by G proteins and contributes to fibrous dysplasia. Proceedings of the National Academy of Sciences. 2011; 108(50):20101-6.
39. Gueorguiev M, Grossman A B. Pituitary gland and β-catenin signaling: from ontogeny to oncogenesis. Pituitary. 2009; 12(3):245-55.
40. Nomura R, Saito T, Mitomi H, Hidaka Y, Lee S-y, Watanabe S, et al. GNAS mutation as an alternative mechanism of activation of the Wnt/β-catenin signaling pathway in gastric adenocarcinoma of the fundic gland type. Human Pathology. 2014; 45(12):2488-96.
41. Wilson C H, McIntyre R E, Arends M J, Adams D J. The activating mutation R201C in GNAS promotes intestinal tumourigenesis in Apc (Min/+) mice through activation of Wnt and ERK1/2 MAPK pathways. Oncogene. 2010; 29(32):4567-75.
42. Tissier F, Cavard C, Groussin L, Perlemoine K, Fumey G, Hagneré A-M, et al. Mutations of β-Catenin in Adrenocortical Tumors: Activation of the Wnt Signaling Pathway Is a Frequent Event in both Benign and Malignant Adrenocortical Tumors. Cancer Research. 2005; 65(17):7622-7.
43. Almeida M Q, Stratakis C A. How does cAMP/protein kinase A signaling lead to tumors in the adrenal cortex and other tissues? Molecular and Cellular Endocrinology. 2011; 336(1-2):162-8.
44. Innamorati G, Wilkie T M, Kantheti H S, Valenti M T, Dalle Carbonare L, Giacomello L, et al. The curious case of Galphas gain-of-function in neoplasia. BMC Cancer. 2018; 18(1):293.
45. Dai S A, Hu Q, Gao R, Lazar A, Zhang Z, von Zastrow M, et al. A GTP-state specific cyclic peptide inhibitor of the GTPase Gαs. bioRxiv. 2020:2020.04.25.054080.
46. Onken M D, Makepeace C M, Kaltenbronn K M, Choi J, Hernandez-Aya L, Weilbaecher K N, et al. Targeting primary and metastatic uveal melanoma with a G&#xa0; protein inhibitor. Journal of Biological Chemistry. 2021; 296.
47. Coles G L, Cristea S, Webber J T, Levin R S, Moss S M, He A, et al. Unbiased Proteomic Profiling Uncovers a Targetable GNAS/PKA/PP2A Axis in Small Cell Lung Cancer Stem Cells. Cancer Cell. 2020; 38(1):129-43.e7.
48. Franken N A, Rodermond H M, Stap J, Haveman J, van Bree C. Clonogenic assay of cells in vitro. Nat Protoc. 2006; 1(5):2315-9.
49. Dobin A, Davis C A, Schlesinger F, Drenkow J, Zaleski C, Jha S, et al. STAR: ultrafast universal RNA-seq aligner. Bioinformatics. 2013; 29(1):15-21.
50. Tischler G, Leonard S. biobambam: tools for read pair collation based algorithms on BAM files. Source Code for Biology and Medicine. 2014; 9(1):13.
51. Harrow J, Frankish A, Gonzalez J M, Tapanari E, Diekhans M, Kokocinski F, et al. GENCODE: the reference human genome annotation for The ENCODE Project. Genome Res. 2012; 22(9):1760-74.
52. Anders S, Pyl P T, Huber W. HTSeq—a Python framework to work with high-throughput sequencing data. Bioinformatics. 2015; 31(2):166-9.
53. Conway T, Wazny J, Bromage A, Tymms M, Sooraj D, Williams E D, et al. Xenome—a tool for classifying reads from xenograft samples. Bioinformatics. 2012; 28(12): i172-8.
54. Yang I S, Son H, Kim S, Kim S. ISOexpresso: a web-based platform for isoform-level expression analysis in human cancer. BMC Genomics. 2016; 17(1):631.
55. Anders S, Huber W. Differential expression analysis for sequence count data. Genome Biol. 2010; 11(10):R106.
56. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
57. Liberzon A, Birger C, Thorvaldsdottir H, Ghandi M, Mesirov J P, Tamayo P. The Molecular Signatures Database (MSigDB) hallmark gene set collection. Cell Syst. 2015; 1(6):417-25.
58. Gao J, Aksoy B A, Dogrusoz U, Dresdner G, Gross B, Sumer S O, et al. Integrative analysis of complex cancer genomics and clinical profiles using the cBioPortal. Sci Signal. 2013; 6(269): pl1.
59. Cerami E, Gao J, Dogrusoz U, Gross B E, Sumer S O, Aksoy B A, et al. The cBio cancer genomics portal: an open platform for exploring multidimensional cancer genomics data. Cancer discovery. 2012; 2(5):401-4.
60. Kimbrel E A, Davis T N, Bradner J E, Kung A L. In vivo pharmacodynamic imaging of proteasome inhibition. Mol Imaging. 2009; 8(3):140-7.

### REFERENCES FOR EXAMPLE 2 AND EXAMPLE 3

1. Ang C S-P, Shen J P, Hardy-Abeloos C J, Huang J K, Ross J S, Miller V A, Jacobs M T, Chen I L, Xu D, Ali S M. Genomic Landscape of Appendiceal Neoplasms. JCO Precision Oncology. 2018; 2:1-18.
2. Marmor S, Portschy P R, Tuttle T M, Virnig B A. The rise in appendiceal cancer incidence: 2000-2009. J Gastrointest Surg. 2015; 19(4):743-50. doi: 10.1007/s11605-014-2726-7. PubMed PMID: 25560182.
3. Mo S, Zhou Z, Ying Z, Dai W, Xiang W, Han L, Li Q, Wang R, Cai G. Epidemiology of and prognostic factors for appendiceal carcinomas: a retrospective, population-based study. Int J Colorectal Dis. 2019; 34(11):1915-24. doi: 10.1007/s00384-019-03387-y. PubMed PMID: 31642969.
4. Society; A C. American Cancer Society. Cancer Facts & FIGS. 20202020.
5. Siegel R L, Miller K D, Goding Sauer A, Fedewa S A, Butterly L F, Anderson J C, Cercek A, Smith R A, Jemal A. Colorectal cancer statistics, 2020. C A Cancer J Clin. 2020; 70(3):145-64. doi: 10.3322/caac.21601. PubMed PMID: 32133645.
6. Malas M A, Sulak O, Gökçimen A, Sari A. Development of the vermiform appendix during the fetal period. Surgical and radiologic anatomy. 2004; 26(3):202-7.
7. Tejani M A, Ter Veer A, Milne D, Ottesen R, Bekaii-Saab T, Benson A B, Schrag D, Shibata S, Skibber J, Weiser M. Systemic therapy for advanced appendiceal adenocarcinoma: an analysis from the NCCN Oncology Outcomes Database for colorectal cancer. Journal of the National Comprehensive Cancer Network. 2014; 12(8):1123-30.
8. Lu P, Fields A C, Meyerhardt J A, Davids J S, Shabat G, Bleday R, Goldberg J E, Nash G M, Melnitchouk N. Systemic chemotherapy and survival in patients with metastatic low-grade appendiceal mucinous adenocarcinoma. J Surg Oncol. 2019; 120(3):446-51. doi: 10.1002/jso.25599. PubMed PMID: 31236958; PMCID: PMC6675644.
9. Gleeson E M, Feldman R, Mapow B L, Mackovick L T, Ward K M, Morano W F, Rubin R R, Bowne W B. derived Pseudomyxoma Peritonei (PMP): Molecular Profiling Toward Treatment of a Rare Malignancy. American journal of clinical oncology. 2017.
10. Riihimäki M, Hemminki A, Sundquist J, Hemminki K. Patterns of metastasis in colon and rectal cancer. Sci Rep. 2016; 6:29765-. doi: 10.1038/srep29765. PubMed PMID: 27416752.
11. Raghav K P, Shetty A V, Kazmi S M, Zhang N, Morris J, Taggart M, Fournier K, Royal R, Mansfield P, Eng C. Impact of molecular alterations and targeted therapy in appendiceal adenocarcinomas. The oncologist. 2013; 18(12):1270-7.
12. Raghav K, Shen J P, Jácome A A, Guerra J L, Scally C P, Taggart M W, Foo W C, Matamoros A, Shaw K R, Fournier K, Overman M J, Eng C. Integrated clinico-molecular profiling of appendiceal adenocarcinoma reveals a unique grade-driven entity distinct from colorectal cancer. British Journal of Cancer. 2020. doi: 10.1038/s41416-020-1015-3.
13. Alakus H, Babicky M L, Ghosh P, Yost S, Jepsen K, Dai Y, Arias A, Samuels M L, Mose E S, Schwab R B. Genome-wide mutational landscape of mucinous carcinomatosis peritonei of appendiceal origin. Genome medicine. 2014; 6(5):43.
14. Fecteau R E, Lutterbaugh J, Markowitz S D, Willis J, Guda K. GNAS mutations identify a set of right-sided, RAS mutant, villous colon cancers. PLOS One. 2014; 9(1): e87966. Epub 2014 Feb. 6. doi: 10.1371/journal.pone.0087966. PubMed PMID: 24498230; PMCID: PMC3907576.
15. Nomura R, Saito T, Mitomi H, Hidaka Y, Lee S Y, Watanabe S, Yao T. GNAS mutation as an alternative mechanism of activation of the Wnt/beta-catenin signaling pathway in gastric adenocarcinoma of the fundic gland type. Hum Pathol. 2014; 45(12):2488-96. doi: 10.1016/j.humpath.2014.08.016. PubMed PMID: 25288233.
16. Ideno N, Yamaguchi H, Ghosh B, Gupta S, Okumura T, Steffen D J, Fisher C G, Wood L D, Singhi A D, Nakamura M, Gutkind J S, Maitra A. GNAS (R201C) Induces Pancreatic Cystic Neoplasms in Mice That Express Activated KRAS by Inhibiting YAP1 Signaling. Gastroenterology. 2018; 155(5):1593-607 e12. doi: 10.1053/j.gastro.2018.08.006. PubMed PMID: 30142336; PMCID: 6219919.
17. Coles G L, Cristea S, Webber J T, Levin R S, Moss S M, He A, Sangodkar J, Hwang Y C, Arand J, Drainas A P, Mooney N A, Demeter J, Spradlin J N, Mauch B, Le V, Shue Y T, Ko J H, Lee M C, Kong C, Nomura D K, Ohlmeyer M, Swaney D L, Krogan N J, Jackson P K, Narla G, Gordan J D, Shokat K M, Sage J. Unbiased Proteomic Profiling Uncovers a Targetable GNAS/PKA/PP2A Axis in Small Cell Lung Cancer Stem Cells. Cancer Cell. 2020; 38(1):129-43 e7. doi: 10.1016/j.ccell.2020.05.003. PubMed PMID: 32531271; PMCID: PMC7363571.
18. O'Hayre M, Degese M S, Gutkind J S. Novel insights into G protein and G protein-coupled receptor signaling in cancer. Current opinion in cell biology. 2014; 27:126-35. Epub 2014 Feb. 11. doi: 10.1016/j.ceb.2014.01.005. PubMed PMID: 24508914; PMCID: PMC4021379.
19. Vogelstein B, Papadopoulos N, Velculescu V E, Zhou S, Diaz L A, Jr., Kinzler K W. Cancer genome landscapes. Science. 2013; 339(6127):1546-58. doi: 10.1126/science.1235122. PubMed PMID: 23539594; PMCID: PMC3749880.
20. Zhao X, Deng P, Iglesias-Bartolome R, Amornphimoltham P, Steffen D J, Jin Y, Molinolo A A, de Castro L F, Ovejero D, Yuan Q, Chen Q, Han X, Bai D, Taylor S S, Yang Y, Collins M T, Gutkind J S. Expression of an active Galphas mutant in skeletal stem cells is sufficient and necessary for fibrous dysplasia initiation and maintenance. Proc Natl Acad Sci USA. 2018; 115(3): E428-E37. doi: 10.1073/pnas.1713710115. PubMed PMID: 29282319; PMCID: PMC5776975.
21. Stein M K, Williard F W, Xiu J, Tsao M W, Martin M G, Deschner B W, Dickson P V, Glazer E S, Yakoub D, Shibata D, Grothey A F, Philip P A, Hwang J J, Shields A F, Marshall J L, Korn W M, Lenz H-J, Deneve J L. Comprehensive tumor profiling reveals unique molecular differences between peritoneal metastases and primary colorectal adenocarcinoma. Journal of Surgical Oncology. 2020; 121(8):1320-8.
22. Warren A, Jones A, Shibue T, Hahn W C, Boehm J S, Vazquez F, Tsherniak A, McFarland J M. Global computational alignment of tumor and cell line transcriptional profiles. bioRxiv. 2020:2020.03.25.008342. doi: 10.1101/2020.03.25.008342.
23. Barretina J, Caponigro G, Stransky N, Venkatesan K, Margolin A A, Kim S, Wilson C J, Lehár J, Kryukov G V, Sonkin D, Reddy A, Liu M, Murray L, Berger M F, Monahan J E, Morais P, Meltzer J, Korejwa A, Jané-Valbuena J, Mapa F A, Thibault J, Bric-Furlong E, Raman P, Shipway A, Engels I H, Cheng J, Yu G K, Yu J, Aspesi P, de Silva M, Jagtap K, Jones M D, Wang L, Hatton C, Palescandolo E, Gupta S, Mahan S, Sougnez C, Onofrio R C, Liefeld T, MacConaill L, Winckler W, Reich M, Li N, Mesirov J P, Gabriel S B, Getz G, Ardlie K, Chan V, Myer V E, Weber B L, Porter J, Warmuth M, Finan P, Harris J L, Meyerson M, Golub T R, Morrissey M P, Sellers W R, Schlegel R, Garraway L A. The Cancer Cell Line Encyclopedia enables predictive modelling of anticancer drug sensitivity. Nature. 2012; 483(7391):603-7. doi: 10.1038/nature11003.
24. Abagyan R A, Totrov M M. Biased probability Monte Carlo as a powerful global energy optimization method for biomolecular structure prediction. Abstr Pap Am Chem S. 1996; 211:35-Comp. PubMed PMID: WOS: A1996UA48202226.
25. Abagyan R, Totrov M, Kuznetsov D. Icm—a New Method for Protein Modeling and Design-Applications to Docking and Structure Prediction from the Distorted Native Conformation. J Comput Chem. 1994; 15(5):488-506. doi: DOI 10.1002/jcc.540150503. PubMed PMID: WOS:A1994NG57200002.
26. Hu Q, Shokat K M. Disease-Causing Mutations in the G Protein Galphas Subvert the Roles of GDP and GTP. Cell. 2018; 173(5):1254-64 e11. doi: 10.1016/j.cell.2018.03.018. PubMed PMID: 29628140; PMCID: PMC5959768.
27. Robins M J, Fouron Y, Mengel R. Nucleic acid related compounds. II. Adenosine 2′, 3′-ribo-epoxide. Synthesis, intramolecular degradation, and transformation into 3′-substituted xylofuranosyl nucleosides and the lyxo-epoxide. The Journal of organic chemistry. 1974; 39(11):1564-70.
28. Shultz S, Worzella T, Gallagher A, Shieh J, Goueli S, Hsiao K, Vidugiriene J. Miniaturized GPCR signaling studies in 1536-well format. J Biomol Tech. 2008; 19(4):267-74. PubMed PMID: 19137117; PMCID: PMC2567138.
29 Buccioni M, Marucci G, Dal Ben D, Giacobbe D, Lambertucci C, Soverchia L, Thomas A, Volpini R, Cristalli G. Innovative functional cAMP assay for studying G protein-coupled receptors: application to the pharmacological characterization of GPR17. Purinergic Signal. 2011; 7(4):463-8. doi: 10.1007/s11302-011-9245-8. PubMed PMID: 21773766; PMCID: PMC3224640.
30. Hu Q, Shokat K M. Disease-causing mutations in the G protein Gαs subvert the roles of GDP and GTP. Cell. 2018; 173(5):1254-64. el1.
31. Lam P C-H, Abagyan R, Totrov M. Macrocycle modeling in ICM: benchmarking and evaluation in D3R Grand Challenge 4. Journal of computer-aided molecular design. 2019; 33(12):1057-69.
32. Meewan I, Zhang X, Roy S, Ballatore C, O'Donoghue A J, Schooley R T, Abagyan R. Discovery of New Inhibitors of Hepatitis C Virus NS3/4A Protease and Its D168A Mutant. ACS omega. 2019; 4(16):16999-7008.
33. Scarpino A, Ferenczy GrG, Keser& GrM. Comparative evaluation of covalent docking tools. Journal of chemical information and modeling. 2018; 58(7):1441-58.
34. Müller M P, Jeganathan S, Heidrich A, Campos J, Goody R S. Nucleotide based covalent inhibitors of KRas can only be efficient in vivo if they bind reversibly with GTP-like affinity. Scientific reports. 2017; 7(1):1-11.
35. Pradere U, Garnier-Amblard E C, Coats S J, Amblard F, Schinazi R F. Synthesis of nucleoside phosphate and phosphonate prodrugs. Chemical reviews. 2014; 114(18):9154-218.
36. Gehringer M, Laufer S A. Emerging and Re-Emerging Warheads for Targeted Covalent Inhibitors: Applications in Medicinal Chemistry and Chemical Biology. J Med Chem. 2019; 62(12):5673-724. Epub 2018 Dec. 20. doi: 10.1021/acs.jmedchem.8b01153. PubMed PMID: 30565923.
37. Bauer R A. Covalent inhibitors in drug discovery: from accidental discoveries to avoided liabilities and designed therapies. Drug discovery today. 2015; 20(9):1061-73.
38. Hong D S, Fakih M G, Strickler J H, Desai J, Durm G A, Shapiro G I, Falchook G S, Price T J, Sacher A, Denlinger C S, Bang Y-J, Dy G K, Krauss J C, Kuboki Y, Kuo J C, Coveler A L, Park K, Kim T W, Barlesi F, Munster P N, Ramalingam S S, Burns T F, Meric-Bernstam F, Henary H, Ngang J, Ngarmchamnanrith G, Kim J, Houk B E, Canon J, Lipford J R, Friberg G, Lito P, Govindan R, Li B T. KRASG12C Inhibition with Sotorasib in Advanced Solid Tumors. New England Journal of Medicine. 2020; 383(13):1207-17. doi: 10.1056/NEJMoa1917239.
39. Yuan C, Chang C T, Axelrod A, Siegel D. Synthesis of (+)-complanadine A, an inducer of neurotrophic factor excretion. Journal of the American Chemical Society. 2010; 132(17):5924-5. Epub 2010 Apr. 15. doi: 10.1021/ja101956x. PubMed PMID: 20387896.
40. Axelrod A, Eliasen A M, Chin M R, Zlotkowski K, Siegel D. Syntheses of xanthofulvin and vinaxanthone, natural products enabling spinal cord regeneration. Angew Chem Int Ed Engl. 2013; 52(12):3421-4. doi: 10.1002/anie.201205837. PubMed PMID: 23086682.
41. Camelio A M, Johnson T C, Siegel D. Total Synthesis of Celastrol, Development of a Platform to Access Celastroid Natural Products. J Am Chem Soc. 2015; 137(37):11864-7. doi: 10.1021/jacs.5b06261. PubMed PMID: 26331410.
42. Johnson T C, Chin M R, Han T X, Shen J P, Rana T, Siegel D. Synthesis of Eupalinilide E, a Promoter of Human Hematopoietic Stem and Progenitor Cell Expansion. Journal of the American Chemical Society. 2016; 138(18):6068-73. doi: 10.1021/jacs.6b03055. PubMed PMID: WOS: 000375889100048.
43. LaMonte G M, Rocamora F, Marapana D S, Gnadig N F, Ottilie S, Luth M R, Worgall T S, Goldgof G M, Mohunlal R, Santha Kumar T R, Thompson J K, Vigil E, Yang J, Hutson D, Johnson T, Huang J, Williams R M, Zou B Y, Cheung A L, Kumar P, Egan T J, Lee MCS, Siegel D, Cowman A F, Fidock D A, Winzeler E A. Pan-active imidazolopiperazine antimalarials target the Plasmodium falciparum intracellular secretory pathway. Nat Commun. 2020; 11(1):1780. Epub 2020 Apr. 15. doi: 10.1038/s41467-020-15440-4. PubMed PMID: 32286267; PMCID: PMC7156427.
44. Antonova-Koch Y, Meister S, Abraham M, Luth M R, Ottilie S, Lukens A K, Sakata-Kato T, Vanaerschot M, Owen E, Jado J C, Maher S P, Calla J, Plouffe D, Zhong Y, Chen K, Chaumeau V, Conway A J, McNamara C W, Ibanez M, Gagaring K, Serrano F N, Eribez K, Taggard C M, Cheung A L, Lincoln C, Ambachew B, Rouillier M, Siegel D, Nosten F, Kyle D E, Gamo F J, Zhou Y, Llinas M, Fidock D A, Wirth D F, Burrows J, Campo B, Winzeler E A. Open-source discovery of chemical leads for next-generation chemoprotective antimalarials. Science. 2018; 362(6419). Epub 2018 Dec. 14. doi: 10.1126/science.aat9446. PubMed PMID: 30523084.
45. Cowell A N, Istvan E S, Lukens A K, Gomez-Lorenzo M G, Vanaerschot M, Sakata-Kato T, Flannery E L, Magistrado P, Owen E, Abraham M, LaMonte G, Painter H J, Williams R M, Franco V, Linares M, Arriaga I, Bopp S, Corey V C, Gnadig N F, Coburn-Flynn O, Reimer C, Gupta P, Murithi J M, Moura P A, Fuchs O, Sasaki E, Kim S W, Teng C H, Wang L T, Akidil A, Adjalley S, Willis P A, Siegel D, Tanaseichuk O, Zhong Y, Zhou Y, Llinas M, Ottilie S, Gamo F J, Lee MCS, Goldberg D E, Fidock D A, Wirth D F, Winzeler E A. Mapping the malaria parasite druggable genome by using in vitro evolution and chemogenomics. Science. 2018; 359(6372):191-9. Epub 2018 Jan. 13. doi: 10.1126/science.aan4472. PubMed PMID: 29326268; PMCID: PMC5925756.
46. Chin M R, Zlotkowski K, Han M, Patel S, Eliasen A M, Axelrod A, Siegel D. Expedited access to vinaxanthone and chemically edited derivatives possessing neuronal regenerative effects through ynone coupling reactions. ACS Chem Neurosci. 2015; 6(4):542-50. doi: 10.1021/cn500237z. PubMed PMID: 25615693.
47. Morten B C, Scott R J, Avery-Kiejda K A. Comparison of Three Different Methods for Determining Cell Proliferation in Breast Cancer Cell Lines. J Vis Exp. 2016(115):54350. doi: 10.3791/54350. PubMed PMID: 27685661.
48. Shen J P, Zhao D, Sasik R, Luebeck J, Birmingham A, Bojorquez-Gomez A, Licon K, Klepper K, Pekin D, Beckett A N, Sanchez K S, Thomas A, Kuo C C, Du D, Roguev A, Lewis N E, Chang A N, Kreisberg J F, Krogan N, Qi L, Ideker T, Mali P. Combinatorial CRISPR-Cas9 screens for de novo mapping of genetic interactions. Nat Methods. 2017; 14(6):573-6. doi: 10.1038/nmeth.4225. PubMed PMID: 28319113; PMCID: PMC5449203.
49. Kopetz S, Desai J, Chan E, Hecht J R, O'Dwyer P J, Lee R J, Nolop K B, Saltz L. PLX4032 in metastatic colorectal cancer patients with mutant BRAF tumors. Journal of Clinical Oncology. 2010; 28(15_suppl):3534-. doi: 10.1200/jco.2010.28.15_suppl.3534.
50. Letai A. Functional precision cancer medicine-moving beyond pure genomics. Nat Med. 2017; 23(9):1028-35. Epub 2017 Sep. 9. doi: 10.1038/nm.4389. PubMed PMID: 28886003.
51. Drost J, Clevers H. Organoids in cancer research. Nat Rev Cancer. 2018; 18(7):407-18. Epub 2018 Apr. 26. doi: 10.1038/s41568-018-0007-6. PubMed PMID: 29692415.
52. Vlachogiannis G, Hedayat S, Vatsiou A, Jamin Y, Fernandez-Mateos J, Khan K, Lampis A, Eason K, Huntingford I, Burke R, Rata M, Koh D M, Tunariu N, Collins D, Hulkki-Wilson S, Ragulan C, Spiteri I, Moorcraft S Y, Chau I, Rao S, Watkins D, Fotiadis N, Bali M, Darvish-Damavandi M, Lote H, Eltahir Z, Smyth E C, Begum R, Clarke P A, Hahne J C, Dowsett M, de Bono J, Workman P, Sadanandam A, Fassan M, Sansom O J, Eccles S, Starling N, Braconi C, Sottoriva A, Robinson S P, Cunningham D, Valeri N. Patient-derived organoids model treatment response of metastatic gastrointestinal cancers. Science. 2018; 359(6378):920-6. doi: 10.1126/science.aao2774. PubMed PMID: 29472484.
53. Ooft S N, Weeber F, Dijkstra K K, McLean C M, Kaing S, van Werkhoven E, Schipper L, Hoes L, Vis D J, van de Haar J, Prevoo W, Snaebjornsson P, van der Velden D, Klein M, Chalabi M, Boot H, van Leerdam M, Bloemendal H J, Beerepoot L V, Wessels L, Cuppen E, Clevers H, Voest E E. Patient-derived organoids can predict response to chemotherapy in metastatic colorectal cancer patients. Science translational medicine. 2019; 11(513). Epub 2019 Oct. 11. doi: 10.1126/scitranslmed.aay2574. PubMed PMID: 31597751.
54. Yao Y, Xu X, Yang L, Zhu J, Wan J, Shen L, Xia F, Fu G, Deng Y, Pan M, Guo Q, Gao X, Li Y, Rao X, Zhou Y, Liang L, Wang Y, Zhang J, Zhang H, Li G, Zhang L, Peng J, Cai S, Hu C, Gao J, Clevers H, Zhang Z, Hua G. Patient-Derived Organoids Predict Chemoradiation Responses of Locally Advanced Rectal Cancer. Cell stem cell. 2020; 26(1):17-26 e6. Epub 2019 Nov. 26. doi: 10.1016/j.stem.2019.10.010. PubMed PMID: 31761724.
55. Ganesh K, Wu C, O'Rourke K P, Szeglin B C, Zheng Y, Sauve C G, Adileh M, Wasserman I, Marco M R, Kim A S, Shady M, Sanchez-Vega F, Karthaus W R, Won H H, Choi S H, Pelossof R, Barlas A, Ntiamoah P, Pappou E, Elghouayel A, Strong J S, Chen C T, Harris J W, Weiser M R, Nash G M, Guillem J G, Wei I H, Kolesnick R N, Veeraraghavan H, Ortiz E J, Petkovska I, Cercek A, Manova-Todorova K O, Saltz L B, Lavery J A, DeMatteo R P, Massague J, Paty P B, Yaeger R, Chen X, Patil S, Clevers H, Berger M F, Lowe S W, Shia J, Romesser P B, Dow L E, Garcia-Aguilar J, Sawyers C L, Smith J J. A rectal cancer organoid platform to study individual responses to chemoradiation. Nat Med. 2019; 25(10):1607-14. Epub 2019 Oct. 9. doi: 10.1038/s41591-019-0584-2. PubMed PMID: 31591597; PMCID: PMC7385919.
56. van de Wetering M, Francies H E, Francis J M, Bounova G, Iorio F, Pronk A, van Houdt W, van Gorp J, Taylor-Weiner A, Kester L, McLaren-Douglas A, Blokker J, Jaksani S, Bartfeld S, Volckman R, van Sluis P, Li V S, Seepo S, Sekhar Pedamallu C, Cibulskis K, Carter S L, McKenna A, Lawrence M S, Lichtenstein L, Stewart C, Koster J, Versteeg R, van Oudenaarden A, Saez-Rodriguez J, Vries R G, Getz G, Wessels L, Stratton M R, McDermott U, Meyerson M, Garnett M J, Clevers H. Prospective derivation of a living organoid biobank of colorectal cancer patients. Cell. 2015; 161(4):933-45. Epub 2015 May 11. doi: 10.1016/j.cell.2015.03.053. PubMed PMID: 25957691; PMCID: PMC6428276.
57. Organoids May Point to Best Therapy. Cancer discovery. 2018; 8(5):524-. doi: 10.1158/2159-8290.cd-nb2018-029.
58. Kuracha M R, Thomas P, Loggie B W, Govindarajan V. Patient-derived xenograft mouse models of pseudomyxoma peritonei recapitulate the human inflammatory tumor microenvironment. Cancer Med. 2016; 5(4):711-9. doi: 10.1002/cam4.640. PubMed PMID: 26833741; PMCID: PMC4831290.
59. Mavanur A A, Parimi V, O'Malley M, Nikiforova M, Bartlett D L, Davison J M. Establishment and characterization of a murine xenograft model of appendiceal mucinous adenocarcinoma. Int J Exp Pathol. 2010; 91(4):357-67. doi: 10.1111/j.1365-2613.2010.00721.x. PubMed PMID: 20586814; PMCID: PMC2962894.
60. Dilly A K, Song X, Zeh H J, Guo Z S, Lee Y J, Bartlett D L, Choudry H A. Mitogen-activated protein kinase inhibition reduces mucin 2 production and mucinous tumor growth. Transl Res. 2015; 166(4):344-54. doi: 10.1016/j.trsl.2015.03.004. PubMed PMID: 25890193.
61. Lin Y-L, Ma R, Li Y. The biological basis and function of GNAS mutation in pseudomyxoma peritonei: a review. Journal of Cancer Research and Clinical Oncology. 2020; 146(9):2179-88. doi: 10.1007/s00432-020-03321-8.

1. A compound of Formula (I) 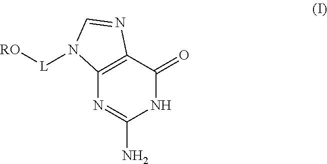
or a pharmaceutically acceptable salt thereof;
wherein:
R is H, 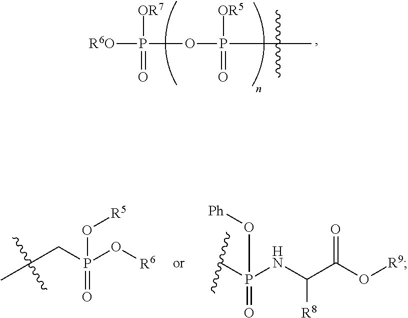
L is 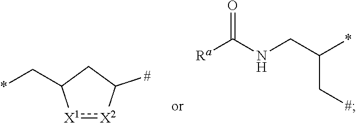
* denotes the linkage point with the —OR group and
#denotes the linkage point with the heterocycle;
X¹ is CR¹ or CR¹R²;
X² is CR³ or CR³R⁴;
- - - is absent or a single bond, provided that when - - - is a single bond, X¹ is CR¹ and X² is CR³;
R¹, R², R³, and R⁴, when present, are each independently H, OH, SH, CN, C₁-C₆ alkyl, C₁-C₆ alkoxy, a —O-sulfolnyl group, or halide; or any two of R¹, R², R³, and R⁴ are taken together with the carbon atoms to which they are bound to form a ring having 3-6 ring atoms comprising a moiety selected from the group consisting of O, S, SO, SO₂, and O(SO₂)O, or if on the same carbon atom, to form an oxo (═O) group or an optionally substituted alkenyl group;
R^{a} is optionally substituted alkenyl or haloalkyl;
R⁵, R⁶, and R⁷ are each independently absent, H, or —(CH₂)—O—(CO)—(C₁-C₆ alkyl);
R⁸ and R⁹ are each independently H or C₁-C₆ alkyl; and
n is 0, 1 or 2.

2. The compound of claim 1, having a structure of Formula (II) 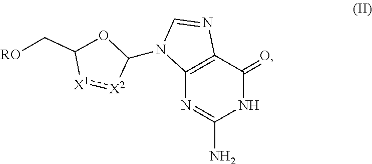
or a pharmaceutically acceptable salt thereof.

3. The compound of claim 1, having a structure of Formula (IIa) 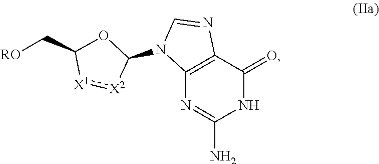
or a pharmaceutically acceptable salt thereof.

4. The compound of claim 1, wherein - - - is absent or a single bond, and, X¹ is CR¹ and X² is CR³.

5. (canceled)

6. The compound of claim 1, wherein R¹, R², R³, and R⁴, when present, are each independently H, OH, SH, a —O-sulfolnyl group, halide, or CN, and optionally wherein the —O-sulfolnyl group is mesylate (—OSO₂Me).

7. (canceled)

8. The compound of claim 1, wherein R¹ and R³ are taken together with the carbon atoms to which they are bound to form a 5-membered ring comprising a O(SO₂)O moiety or wherein R¹ and R³ are taken together with the carbon atoms to which they are bound to form an epoxide group.

9. (canceled)

10. The compound of claim 1, wherein R¹ and R², or R³ and R⁴ are taken together with the carbon atom to which they are bound to form an epoxide group or wherein R¹ and R², or R³ and R⁴ are taken together with the carbon atom to which they are bound to form an oxo (═O) group or an optionally substituted alkenyl group.

11. (canceled)

12. The compound of claim 10, wherein the alkenyl group is ═CH₂.

13. The compound of claim 1 any one of claims 1-12, wherein R¹, R², R³, and R⁴, when present, then at least one of R¹, R², R³, and R⁴ is not H or wherein R¹, R², R³ and R⁴, when present, then at least one of R¹ and R² is not H, and, at least one of R³ and R⁴ is not H.

14. (canceled)

15. The compound of claim 4, wherein at least one of R¹ and R³ is not H.

16. The compound of claim 1, having a structure of selected from Formula (III) 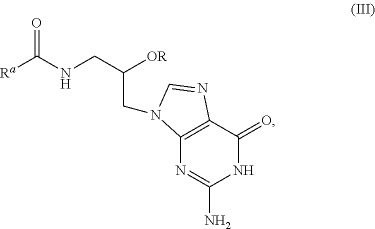
or a pharmaceutically acceptable salt thereof;
Formula (IIIa) or (IIIb) 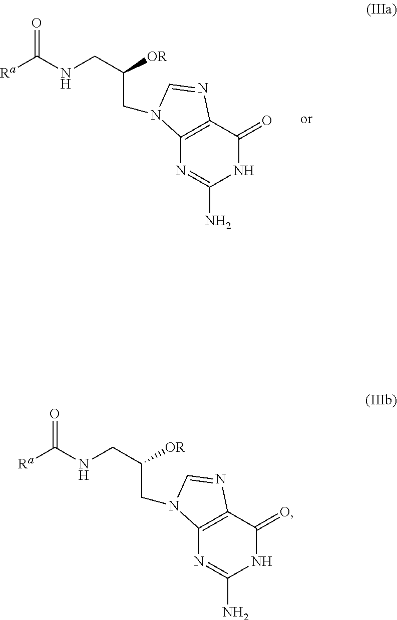
or a pharmaceutically acceptable salt thereof.

17. (canceled)

18. The compound of claim 1, wherein R^{a} is

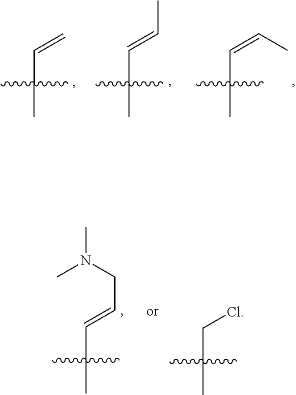

19. The compound of claim 1, wherein n is 0 or 1.

20. The compound of claim 1, wherein R is H, PO₃²⁻, PO₃PO₃³⁻,

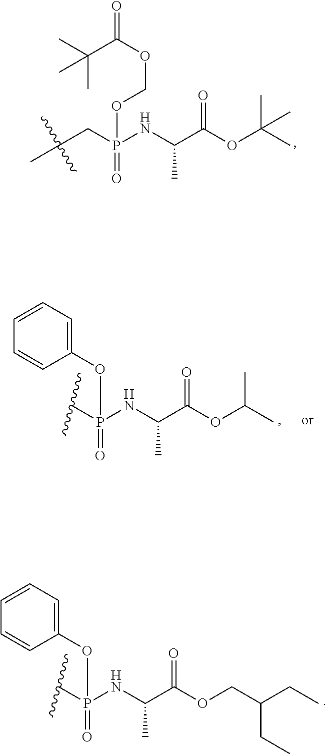 [SMILES: *CP(=O)(N[C@@H](C)C(=O)OC(C)(C)C)OCOC(=O)C(C)(C)C ; *P(=O)(N[C@@H](C)C(=O)OC(C)C)Oc1ccccc1 ; *P(=O)(N[C@@H](C)C(=O)OCC(CC)CC)Oc1ccccc1]

21. The compound of claim 1, having the following structures: 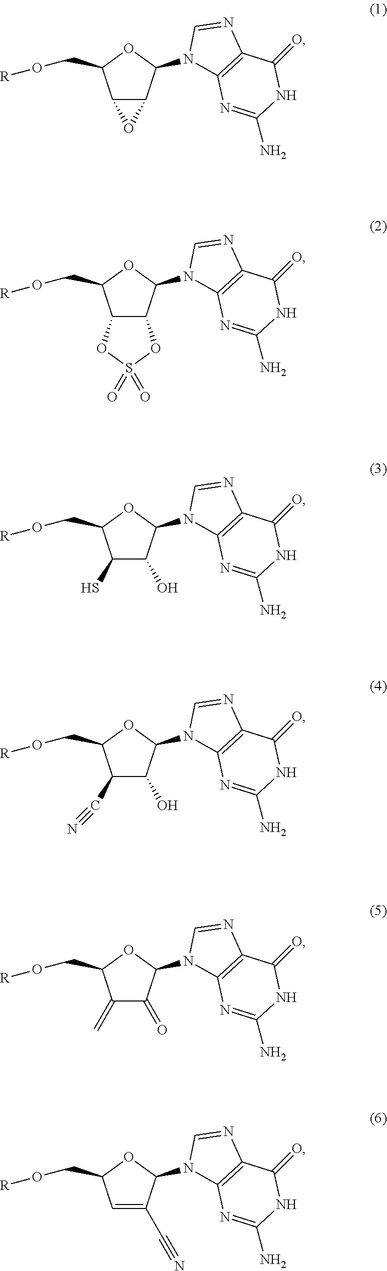 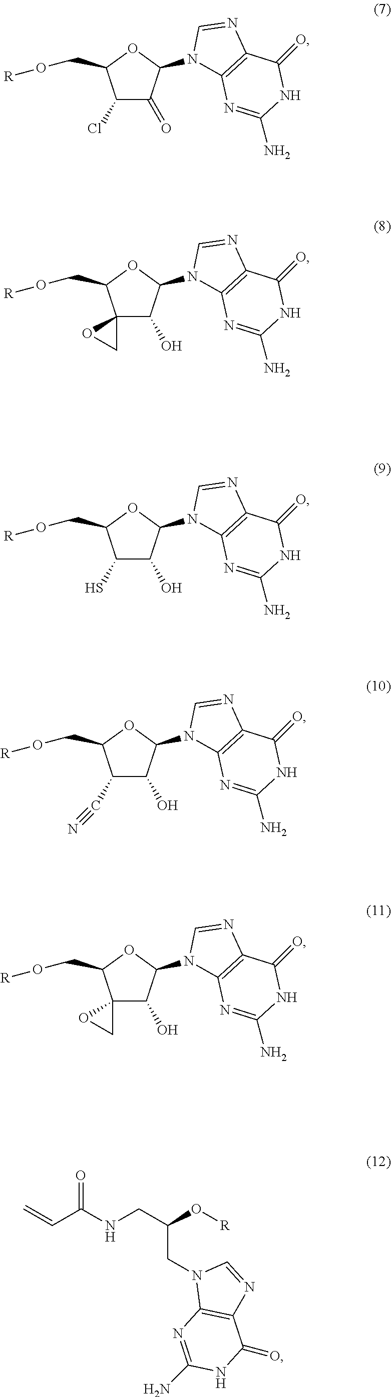 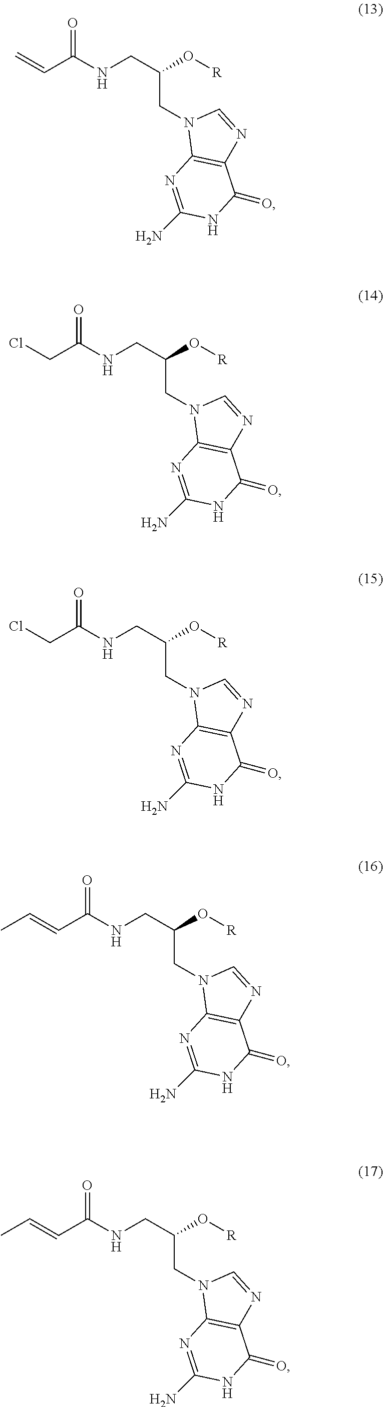 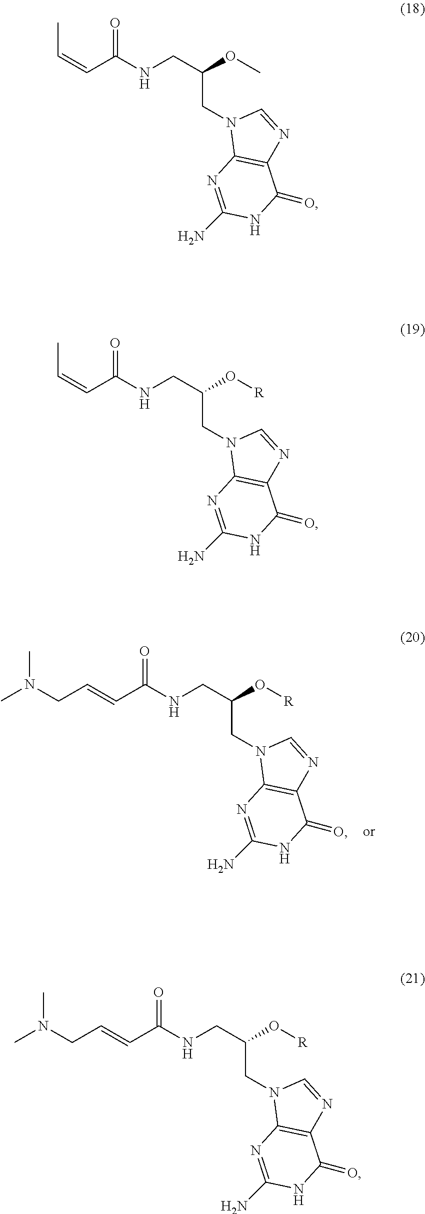
or a pharmaceutically acceptable salt thereof.

22. The compound of claim 1, having the following structure: 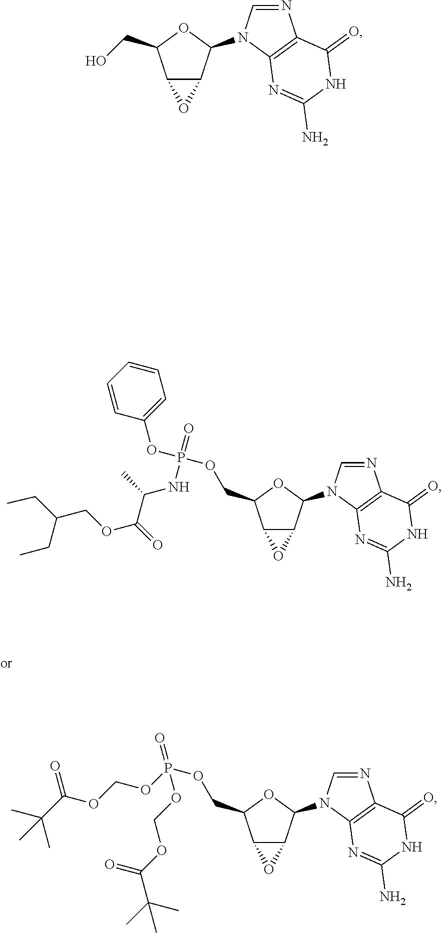 [SMILES: Nc1nc2c(ncn2[C@@H]2O[C@H](CO)[C@H]3O[C@H]32)c(=O)[nH]1 ; CCC(CC)COC(=O)[C@H](C)NP(=O)(OC[C@H]1O[C@@H](n2cnc3c(=O)[nH]c(N)nc32)[C@@H]2O[C@@H]21)Oc1ccccc1 ; CC(C)(C)C(=O)OCOP(=O)(OCOC(=O)C(C)(C)C)OC[C@H]1O[C@@H](n2cnc3c(=O)[nH]c(N)nc32)[C@@H]2O[C@@H]21]
or a pharmaceutically acceptable salt thereof.

23. A composition comprising the compound of claim 1, or a pharmaceutical acceptable salt thereof.

24. (canceled)

25. A method for one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell, the method comprising contacting the cell with the compound of claim 1.

26-33. (canceled)

34. A method of one or more of: inhibiting the growth of a diseased cell mediated by GNAS activity; inhibiting the growth of a cancer cell, inhibiting the growth of a cancer cell mediated by GNAS activity, or assaying for inhibitory activity of a compound or pharmaceutical activity of a diseased cell in a subject in need thereof, the method comprising administering to the subject an effective amount of the compound of claim 1.

35-44. (canceled)
